(19) United States
(12) Patent Application Publication
(10) Pub. No.: US 2006/0045906 A1
(43) Pub. Date: Mar. 2, 2006

(54) Compositions and methods for activating protein synthesis and deactivating catabolic processes in skeletal muscle

(76) Inventors: Paul T. Gardiner, Mississauga (CA); Marvin A. Heuer, Etobicoke (CA)

Correspondence Address: KENYON & KENYON, ONE BROADWAY, NEW YORK, NY, 10004, US

(21) Appl. No.: 11/211,929

(22) Filed: Aug. 24, 2005

Related U.S. Application Data
(60) Provisional application No. 60/604,534, filed on Aug. 25, 2004.

(51) Int. Cl. A61K 47/00; A61K 31/205

(52) U.S. Cl. 424/439; 514/554

(57) ABSTRACT

A method for activating the protein synthesis machinery and deactivating catabolic processes within skeletal muscle by regulating molecular signals to control anabolic and anti-catabolic activity in skeletal muscle via nutrients including but not limited to amino acids and growth factors. Also provided is a supplemental dietary composition that may include L-Leucine, including salts or derivatives thereof, L-phenylalanine, including salts or derivatives thereof, and/or creatine, including salts or derivatives therof, and may also include sources of dietary protein and/or carbohydrates.

### RELATED APPLICATIONS

This application is based on and claims the benefit of priority to U.S. Provisional Patent Application No. 60/604,534, filed Aug. 25, 2004, which is incorporated by reference herein in its entirety.

### FIELD OF THE INVENTION

The present invention relates to the retention of creatine within the body, and relates in particular but not exclusively to a method and supplement for increasing creatine accumulation in humans. More specifically, the present invention relates to a supplemental dietary composition for activating the protein synthesis machinery and deactivating catabolic processes within skeletal muscle by regulating the molecular signals to control anabolic and anti-catabolic activity in skeletal muscle including, for instance, leucine. In addition, the present invention relates to a method for activating the protein synthesis machinery and deactivating catabolic processes within skeletal muscle by regulating the molecular signals to control anabolic and anti-catabolic activity in skeletal muscle, e.g., by consuming a supplemental dietary composition that includes, for instance, leucine. In addition, the present invention relates to a method of manufacturing a supplemental dietary composition.

### BACKGROUND

Creatine is known to be present in the muscles of vertebrates. It is present in a phosphorylated and a non-phosphorylated form and it is involved in muscular contraction and the development of fatigue. Creatine is produced naturally by the body, but is also obtained from animal foods.

Most bodily creatine is present in muscle, and it is believed that increasing the amount of creatine within muscle favorably affects muscular performance and the amount of work that can be done by the muscle. It has been widely reported that elevating the muscle total creatine store can enhance performance during high-intensity exercise. Accordingly, creatine supplementation has become popular among athletes wishing to improve athletic performance. It is also possible that creatine supplementation may be of therapeutic benefit for patients with muscular and neurological disorders.

Most of the body creatine pool is restricted to skeletal muscle, where it plays a pivotal role in maintaining energy homeostasis. The muscle total creatine store (phosphocreatine and free creatine) in healthy, nonvegetarian subjects is, on average, about 124 mmol/kg dry mass (dm), but it can vary widely among individuals from about 100 to about 150 mmol/kg dm.

Dietary creatine supplementation produces a 20-50% increase in human skeletal muscle total creatine (phosphocreatine and free creatine) stores and parallel biochemical and functional improvements during contraction. See Harris R C, et al. (1992). Clin. Sci.; 83 (3): 367-74; Greenhaff et al. (1994) Am J Physiol; 266 (5): E725-30; (Greenhaff et al (1993), Clin Sci (Loud); 84(5): 565-71, which are incorporated by reference herein in their entirety. Dietary creatine supplementation at a rate of 20 g/day for 5 days has been shown to increase muscle total creatine content by 20% on average. A similar, but more gradual, increase can be obtained when creatine is ingested at a rate of 2 g/day for 28 days. Furthermore, the magnitude of these improvements appears to be directly related to the extent of creatine accumulation. See Greenhaff et al. (1994) supra and Casey et al. (1996) Am J Physiol; 271 (1): E31-7 incorporated by reference herein in its entirety.

Although some individuals are resistant to creatine accumulation, ingesting a sizeable load of simple carbohydrate (100 g carbohydrant/5 g creatine) increased creatine accumulation in all but this load was close to the limit of palatability. (Green et al. (1996) Am J Physiol; 271 (Endocrinol. Metab, 34): E821-6; and, Steenge et al. (1998) Am J Physiol; 275 (38): E974-9 which are both incorporated by reference herein in their entirety. Subsequently it was demonstrated that a supplement comprising 50 g carbohydrate load to (to increase palatability), and 50 g of milk protein, produced nearly the same whole body creatine retention as a supplement comprising 100 g carbohydrate. See Steenge et al, (2000) J Appl Physiol; 89: 1165-71, incorporated by reference herein in its entirety. The increase in creatine accumulation with carbohydrate is believed to result from insulin stimulated creatine transport.

U.S. Pat. No. 5,968,900, incorporated herein by reference in its entirety discloses compositions, which promote increased creatine retention and/or glycogen storage in muscle. The composition comprises creatine or its derivative and a carbohydrate or its derivative. The carbohydrate is in an amount by weight that is greater than the amount of creatine. The amount of carbohydrate and the amount of creatine are effective for increasing creatine retention and/or glycogen storage in muscle. The compositions may be in the form of a pharmaceutical or a dietary supplement and are intended for use in the human or animal body. Other compositions comprise creatine or an active derivative together with insulin or an active derivative. The amount of creatine and the amount of insulin are effective for increasing creatine retention and/or glycogen storage in muscle. The compositions including creatine and insulin may further contain a carbohydrate or its derivative. A method of increasing creatine retention in a human or animal body comprises causing an increase in blood plasma creatine concentration and causing a substantially simultaneous increase in blood plasma insulin concentration. A method of increasing glycogen storage in a human or animal body comprises causing an increase in blood plasma creatine carbohydrate concentration and causing a substantially simultaneous increase in blood plasma creatine concentration. The compositions to increase the creatine retention and/or glycogen storage in the muscle are administered by injection or ingestion.

U.S. Pat. No. 6,479,069, incorporated herein by reference in its entirety, allegedly discloses compositions to meet the needs of individuals, including humans and pets. Nutritional beverages, powders to make the same, a pudding and a nutritional bar are allegedly disclosed whose compositions include the R-□-lipoic acid in the amount of 0.12 grams to 1.5 grams and L-carnitine in the amount of 0.12 grams to 3 grams in addition to the usual composition. Optionally, effective amounts of coenzyme Q and/or creatine also are added. These additional components allegedly fight age-related declines in mitochondrial function which result in less energy and other signs of aging.

U.S. Pat. No. 6,426,361, incorporated herein by reference in its entirety, describes a method for increasing the synthesis and accumulation of beta-alanylhistidine dipeptides, with a simultaneous increase in the accumulation of creatine, in bodily tissues of humans and animals is described. Allegedly this is accomplished by causing an increase in the blood plasma concentrations of beta-alanine and creatine, or the blood plasma concentrations of beta-alanine, L-histidine and creatine, by the ingestion or infusion of a composition including beta-alanine, beta-alanine and creatine, or beta-alanine, L-histidine and creatine, or active derivatives thereof.

U.S. Pat. No. 6,172,114, incorporated herein by reference in its entirety, refers to a creatine supplement comprising creatine and ribose in a pharmaceutically acceptable vehicle for internal administration. The supplement further includes nutrients selected from the group consisting of vitamins, minerals, amino acids and liquid carbohydrates. In addition, the supplement includes a suitable pharmaceutical excipient selected from the group consisting of fillers, lubricants, binders, colorings and flavorings. Further, the supplement is in a pharmaceutical carrier selected from the group consisting of a tablet, capsule, cream, ointment, solution, gel, suspension, suppository or spray. Finally, the creatine in said supplement is creatine monohydrate.

U.S. Pat. No. 5,773,473, incorporated herein by reference in its entirety, refers to a creatine supplement, which contains a combination of creatine and propylene glycol. The supplement preferably contains from about 25-50% creatine and from about 50-75% propylene glycol. The propylene glycol allegedly not only makes the supplement more bioavailable than conventional creatine supplements, but also decreases the incidence of side effects.

U.S. Pat. No. 5,726,146, incorporated herein by reference in its entirety, allegedly describes a dietary supplement formulation which increases lean body mass without concomitant increase of body fat mass, an effect parallel to that seen with usage of synthetic anabolic steroidal compounds but without adverse side-effects. The formulation composition of the invention comprises creatine, taurine, ribonucleic acid, and optimally, a carbohydrate (starch or a simple saccharide) component for enhancing cellular uptake. Other components such as alpha-ketoglutaric acid and salts thereof, and beta-hydroxy-beta-methyl butyric acid and salts thereof can be added for optimal results. The composition may be taken alone or in combination with a nutrient base, which typically includes protein source(s), carbohydrate(s), vitamin(s), and mineral(s) and other amino acids such as L-Glutamine and other natural L-form non-branched chain or branched chain amino acids. Actual studies in weight trained men show statistically significant increases in lean body mass yet with decreases in fat mass within 28 days.

U.S. Pat. No. 5,397,786, incorporated herein by reference in its entirety, refers to a liquid composition to be used as a rehydration drink, particularly suited for the administration to people who do heavy work under severe conditions, e.g. at high temperatures, and to sports people and athletes, as well as to patients who exhibit dehydration symptoms due to severe illnesses such as diarrhea or vomiting, which contains per serving unit water at least 1 to 100 g of at least one carbohydrate, such as glucose polymers, maltodextrin and fructose; 2 to 2500 mg of at least one electrolyte, such as an alkali and/or earth alkali salt; 0.1 to 750 mg of at least one ammonia neutralizer, such as D,L-magnesium aspartate, L-arginine and glutamate; at least one energy enhancer, such as members of the vitamin B group and branched chain amino acids; at least one antioxidant such as .beta.-carotene, vitamin C, vitamin E and selenium; 1 to 30 mg of at least one membrane stabilizer, such as choline chloride, betaine chloride and methionine; and 1 to 200 .mu.g of at least one neuromuscular function enhancer such as octacosanol.

U.S. Pat. No. 5,925,378 refers to a method for enhancing a stable concentration of cellular creatine in a human which includes dissolving an effervescent containing an acidic edible salt form of creatine in water. Once the mixture has completely dissolved the solution is immediately ingested, and an effective amount of creatine is absorbed. Preferably, the effervescent is in the form of a tablet that contains creatine in the form of an edible salt, a mixture of acids, and sodium.

U.S. Pat. Nos. 6,080,788 and 6,232,346 refer to a dietary supplement comprising L-Carnitine (or its functional analogues such as Acetyl-Carnitine or Proprionyl-l-Carnitine), Coenzyme Q10 and Taurine for the correction of the abnormality in mitochondrial energetics in cardiac failure and certain other diseases. A high protein nutritional feeding supplementation with Cysteine, Creatine, Vitamin E (RRR-d-alpha-tocopherol), Vitamin C (ascorbic acid), Selenium, and Thiamin in may be added.

U.S. Pat. No. 6,399,661 describes an oral creatine supplement and the method of making this supplement which includes mixing an alkaline powder with a powdered creatine until the pH of the mixture is in the range between 7-14. A powdered additive is added to the mixture for improving sweetness and taste. Finally, a further alkaline powder is added to the mixture to adjust the pH of the mixture to a range between 7-14. This mixture is then mixed with water prior to ingestion.

U.S. Patent Application Publication No. 20030224062, incorporated herein by reference in its entirety, refers to a dietary or food supplement for healthy humans that includes a combinations of 4-hydroxyisoleucine and creatine, or nutraceutically acceptable derivatives of these two compounds. The supplement may include additives such as carbohydrates or amino acids. The invention further includes a regimen for supplementing a healthy athlete's diet by administering on a regular basis to the athlete 4-hydroxyisoleucine and creatine, or nutraceutically acceptable derivatives of these two compounds. The invention also provided a method for enhancing the body's absorption and utilization of a nutrient, comprising administering a 4-hydroxyisoleucine or a nutraceutically acceptable derivative thereof in combination with the nutrient.

### SUMMARY OF THE INVENTION

The present invention provides a method for activating the protein synthesis machinery and deactivating catabolic processes within skeletal muscle by regulating molecular signals to control anabolic and anti-catabolic activity in skeletal muscle via nutrients including but not limited to amino acids and growth factors. For example, the present invention may provide, by the consumption of a supplemental dietary composition as set forth herein, a method for stimulating muscle growth, increasing muscle mass, increasing weight gain, decreasing muscle catabolism and associated muscle and weight loss, increasing performance, improving body composition, treating muscle wasting or degenerative disease, suppressing the effects of sarcopenia in the aging population and/or providing a beneficial effect by influencing the genetic control system for global protein synthesis.

The present invention also provides for a method of supplementing the diet of an animal, comprising administering to the animal a serving of a low carbohydrate creatine supplement comprising creatine, carbohydrate, protein and one or more naturally occurring free amino acids.

The present invention also provides a supplemental dietary composition that may include L-Leucine, including salts or derivatives thereof, L-phenylalanine, including salts or derivatives thereof, and/or creatine, including salts or derivatives therof, and may also include sources of dietary protein and/or carbohydrates. The supplemental dietary composition may also include one or more of dextrose, alpha-lipoic acid (“ALA”), maltodextrin, WPC-80, bitter blocker flavor, citric acid, banana flavor, potassium citate, sucralose, pineapple flavor and FD&C Yellow #5. The supplemental dietary composition may activate the protein synthesis machinery and deactivate catabolic processes within skeletal muscle by regulating molecular signals to control anabolic and anti-catabolic activity in skeletal muscle. In doing so, the supplemental dietary composition may stimulate muscle growth, increase muscle mass, increase weight gain, decrease muscle catabolism and associated muscle and weight loss, increase performance, improve body composition, treat muscle wasting or degenerative disease, suppress the effects of sarcopenia in the aging population and/or provide a beneficial effect by influencing the genetic control system for global protein synthesis.

In addition, the present invention provides a low carbohydrate creatine supplement comprising; creatine, carbohydrate, protein and a naturally occurring free amino acid wherein a serving of the supplement is effective in increasing creatine accumulation in skeletal muscle.

The present invention also provides for a method of increasing creatine accumulation in skeletal muscle of an animal comprising the steps of: administering a low carbohydrate creatine supplement comprising a serving of creatine, carbohydrate, protein and one or more naturally occurring free amino acids; and increasing the total muscle creatine in the skeletal muscle of an animal.

In addition, the present invention relates to a method of manufacturing a supplemental dietary composition that may activate the protein synthesis machinery and deactivate catabolic processes within skeletal muscle by regulating molecular signals to control anabolic and anti-catabolic activity in skeletal muscle, and in doing so, may stimulate muscle growth, increase muscle mass, increase weight gain, decrease muscle catabolism and associated muscle and weight loss, increase performance, improve body composition, treat muscle wasting or degenerative disease, suppress the effects of sarcopenia in the aging population and/or provide a beneficial effect by influencing the genetic control system for global protein synthesis. In one embodiment, the method of manufacturing a supplemental dietary composition includes the step of mixing one or more of L-Leucine, including salts or derivatives thereof, L-phenylalanine, including salts or derivatives thereof, and creatine, including salts or derivatives therof. The method of manufacturing a supplemental dietary composition may also include the step of mixing one or more of dextrose, ALA, maltodextrin, WPC-80, bitter blocker flavor, citric acid, banana flavor, potassium citate, sucralose, pineapple flavor and FD&C Yellow #5.

The present invention also provides for a method for manufacturing a low carbohydrate creatine supplement comprising; creatine, carbohydrate, protein and a naturally occurring free amino acid the method comprising the following steps: premixing microcrystalline cellulose with the following ingredients to the premix; creatine, dextrose, high quality milk proteins, L-Phenylalanine, L-Leucine, and microcrystalline cellulose; adding magnesium stearate and silica which had been pre-sifted; blending and mixing for 30 minutes; and checking for uniformity/homogeneity and then aliquoting into a serving.

### DESCRIPTION OF THE FIGURES

FIG. 1 is a diagram that illustrates serum insulin concentration (mU/l) following the first oral challenge with Creatine (c), Carbohydrate (CHO), and Protein/Amino Acids and Carbohydrate (PAC), in accordance with various embodiments of the present invention.

FIG. 2 is a diagram that illustrates serum insulin concentration (mU/l) following the third oral challenge with C, CHO, and PAC.

FIG. 3 is a diagram that illustrates serum insulin area under the concentration time curve for 80 min following the first oral challenge with C, CHO, and PAC.

FIG. 4 is a diagram that illustrates serum insulin area under the concentration time curve for 180 min following the first oral challenge with C, CHO, and PAC.

FIG. 5 is a diagram that illustrates serum insulin area under the concentration time curve for 80 min following the third oral challenge with C, CHO and PAC. I

FIG. 6 is a diagram that illustrates serum insulin area under the concentration time curve for 180 min following the first oral challenge with C, CHO and PAC.

FIG. 7 is a diagram that illustrates plasma creatine concentration (μmol/l) following the first oral challenge with C, CHO and PAC.

FIG. 8 is a diagram that illustrates plasma creatine concentration (μmol/l) following the third oral challenge with C, CHO and PAC.

FIG. 9 is a diagram that illustrates plasma creatine AUC (μmol/l/min) 80 min following the first and third oral challenge with C, CHO and PAC.

FIG. 10 is a diagram that illustrates plasma creatine AUC (μmol/l/min) 180 min following the first and third oral challenge with C, CHO and PAC.

FIG. 11 is a diagram that illustrates urinary creatine excretion (mg) 0-24 h.

FIG. 12 is a diagram that illustrates urinary creatine excretion (mg) 24-48 h following administration.

FIG. 13 is a diagram that illustrates urinary creatine excretion (mg) 0-48 h following supplementation.

FIG. 14 is a diagram that illustrates the signaling events involved in the stimulation of translation initiation, according to various embodiments of the present invention.

### DETAILED DESCRIPTION

Conventionally, amino acids have been seen as precursors of protein synthesis. New research has now demonstrated that key amino acids, e.g., leucine and phenylalanine, play an important role as nutrient signals which facilitate protein synthesis via mechanisms such as stimulating insulin release which in turn translates to positive influences on muscle growth and inhibition of muscle breakdown; and or directly activating molecules involved in protein synthesis. Insulin production via key components, as set forth in the present invention, in conjunction with the direct signaling effect of critical amino acids, as set forth in the present invention, work together to directly modify critical control points in muscle to activate the protein kinase mTOR (mammalian target of rapamycin), a site of integration of signals that stimulates muscle protein synthesis. Leucine is a key component in this formula noting that it has been found to be the most potent branch chain amino acid for stimulating muscle protein synthesis. There are also mediated effects via rapamycin independent mechanism. More specifically, both leucine and phenylalanine also may work via indirect mechanisms to augment protein synthesis via multiple pathways. This anabolic signal, in combination with the known benefits of creatine supplementation, is believed to have an additive affect on changing body composition, e.g., weight loss, and athletic performance, by the addition of lean mass.

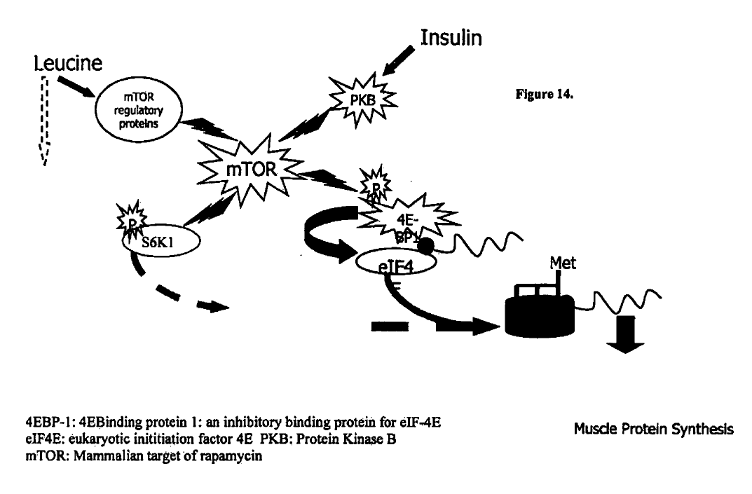

Using leucine, leucine AKG, Leucine ethyl ester, N-acetyl-leucine, nor-leucine salts or other derivatives or bound forms of leucine, with or without the addition of simple sugars, ALA, maltodextrin, carbohydrates or proteins, can elicit an insulin spike, that in turn causes the triggering of protein synthesis pathways that can stimulate the initiation of mRNA translation for muscle growth. Using leucine, leucine AKG, Leucine ethyl ester, N-acetyl-leucine, nor-leucine, salts or other derivatives or bound forms of leucine, with or without the addition of simple sugars, ALA, maltodextrin, carbohydrates or proteins, e.g., whey protein concentrate, also may stimulate protein synthesis through pathways that are independent and/or syngergestic with the pathways that are stimulated through insulin. Using phenylalanine, phenylalanine AKG, phenylalanine ethyl ester, N-acetyl-phenylalanine, salts or any other derivatives or bound forms of phenylalanine, with or without the addition of simple sugars, ALA, maltodextrin, carbohydrates or proteins, can also elicit an insulin spike, that in turn causes the triggering of protein synthesis pathways that can stimulate the initiation of mRNA translation for muscle growth. FIG. 14 illustrates how both phenylalanine (through stimulation of insulin secretion) and leucine activate mTOR which trigger the phosphorylation of 4E-BP1 and S6k1 (and other key protein kinases, i.e. p70S6K), leading to the release of eIF4E (enhancing association of eIF4E with eIF4G) and ultimately leading to increased protein synthesis and inhibition of protein breakdown. It is also illustrated that leucine and phenylalanine directly and indirectly, also may have independent and syngergestic affects on protein synthesis, that utilize a different pathway than the insulin mediated pathway previously described and thus providing method and supplement for enhancing protein synthesis and increasing creatine accumulation/retention in humans.

In an embodiment, the present invention provides a method for increasing lean body mass and improving body composition and athletic performance by regulating the molecular signals that regulate anabolic and anticatabolic activity in skeletal muscle via nutrients, including but not limited to L-leucine, salts and derivatives thereof, L-phenylalanine, salts and derivatives thereof and creatine and derivatives thereof. The above ingredients may be combined with sources of dietary protein and/or carbohydrate.

For example, the present invention, according to various embodiments thereof, provides a supplemental dietary composition that may include L-Leucine, including salts or derivatives thereof, L-phenylalanine, including salts or derivatives thereof, and/or creatine, including salts or derivatives therof, and may also include sources of dietary protein and/or carbohydrates. The supplemental dietary composition may activate the protein synthesis machinery and deactivate catabolic processes within skeletal muscle by regulating molecular signals to control anabolic and anti-catabolic activity in skeletal muscle, and in doing so, may stimulate muscle growth, increase muscle mass, increase weight gain, decrease muscle catabolism and associated muscle and weight loss, increase performance, improve body composition, treat muscle wasting or degenerative disease, suppress the effects of sarcopenia in the aging population and/or provide a beneficial effect by influencing the genetic control system for global protein synthesis.

In an embodiment of the present invention, which is set forth in greater detail in Example 1 below, the supplemental dietary composition may include maltodextrin, creatine monohydrate, whey protein isolate, taurine, citric acid, flavoring, alpha lipoic acid, ascorbic acid, dipotassium phosphate, magnesium phosphate, tricreatine malate, dicreatine malate, L-Leucine, L-Phenylalanine, disodium phosphate, betain, acesulfame potassium, sucralose, coloring, fenugreek extract, D-pinitol and/or chromium polynicotinate.

In the embodiment set forth in Example 3, the supplemental dietary composition includes maltodextrin, creatine monohydrate, whey protein isolate, taurine, citric acid, flavoring, alpha lipoic acid, dipotassium phosphate, magnesium phosphate, tricreatine malate, dicreatine malate, L-Leucine, L-Phenylalanine, disodium phosphate, betain, acesulfame potassium, sucralose and coloring.

In the embodiment set forth in Example 5, the supplemental dietary composition includes dextrose, maltodextrin, partly hydrolyzed whey protein, L-Leucine, L-Phenylalanine, creatine monohydrate, xanthan gum, flavoring and coloring.

In the embodiment set forth in Example 7, the supplemental dietary composition includes dextrose, maltodextrin, WPC-80, L-Leucine, L-Phenylalanine, creatine monohydrate, bitter blocker flavor, citric acid, banana flavor, potassium citrate, sucralose, pineapple flavor and FD&C Yellow #5. In the embodiment set forth in Examples 8 and 9, the supplemental dietary composition includes dextrose, maltodextrin, WPC-80, L-Leucine, L-Phenylalanine, creatine monohydrate, alpha-lipoic acid, bitter blocker flavor, citric acid, banana flavor, potassium citrate, sucralose, pineapple flavor and FD&C Yellow #5. In the embodiment set forth in Example 10, the supplemental dietary composition includes maltodextrin, WPC-80, L-Leucine, L-Phenylalanine, creatine monohydrate, bitter blocker flavor, citric acid, banana flavor, potassium citrate, sucralose, pineapple flavor and FD&C Yellow #5.

The present invention also provides a low carbohydrate creatine supplement comprising; creatine, carbohydrate, protein and a naturally occurring free amino acid wherein a serving of the supplement is effective in increasing creatine accumulation in skeletal muscle.

The present invention may also provide a method of activating the protein synthesis machinery and deactivating catabolic processes within skeletal muscle by regulating molecular signals to control anabolic and anti-catabolic activity in skeletal muscle, and in doing so, may provide a method for stimulating muscle growth, increasing muscle mass, increasing weight gain, decreasing muscle catabolism and associated muscle and weight loss, increasing performance, improving body composition, treating muscle wasting or degenerative disease, suppressing the effects of sarcopenia in the aging population and/or providing a beneficial effect by influencing the genetic control system for global protein synthesis. For example, the method may include the consumption of the supplemental dietary composition according to any of the various embodiments of the present invention set forth herein. Advantageously, consumption of the supplemental dietary composition is combined with a reduced calorie diet and a program of regular exercise.

As set forth above, the use of, e.g., L-Leucine, including salts or derivatives thereof, L-phenylalanine, including salts or derivatives thereof, and/or creatine, including salts or derivatives therof, and may also include sources of dietary protein and/or carbohydrates, as set forth in the example embodiments above, may provide various effects or benefits. For example, the supplemental dietary composition may perform, provide or enable one or more of the following: muscle gene expression activator; switch off catabolism; stimulates gene expression for muscle growth; directly promotes muscle protein synthesis; turns on muscle promoting pathways; stimulates muscle growth; stimulates/Initiates mRNA translation for muscle growth; accelerates muscle protein synthesis; activates mTOR expression to turn on protein synthesis; intracellular regulation of protein building; optimizes muscle accretion; regulates signaling mechanisms to promote anabolism; regulates signaling mechanisms to inhibit catabolism; phosphorylates key proteins involved in regulating muscle growth; reach your full genetic potential; reach max protein synthesis rates; break through your genetic barriers; optimizes muscle growth; genetic manipulation for advanced muscle growth; genetically manipulates molecular mechanism for muscle growth; genetically enhanced muscle building; gene powered muscle building; genetically induced muscle growth; genetically stimulated muscle building; genetic muscle promoter; regulates skeletal muscle growth; stimulates muscle development; mediates skeletal muscle homeostasis; regulates muscle's genetic potential; genetic muscle growth stimulator; genetically optimized muscle building; stimulates gene expression for muscle growth; directly promotes muscle protein synthesis; turns on muscle promoting pathways; muscle growth activator; direct muscle growth stimulator; potent anabolism promoter; intense anabolic signaling agent; pushes you past your genetic potential; directly turns on anabolic switches in muscles; potently enhances muscle growth; directly activates muscle building pathways; regulates anabolic mechanisms in muscles; most powerful anabolic nutrient/molecule; optimizes muscle protein synthesis; revs-up anabolic signaling at the molecular level; intense protein synthesis stimulation; serious anabolic nutrient signaling; genetically induced muscle hypertrophy; genetically enhances muscle strength; and genetic control over muscle growth.

According to various embodiments of the present invention, the supplemental dietary composition may be consumed in any form. For instance, the dosage form of the supplemental dietary composition may be provided as, e.g., a powder beverage mix, a liquid beverage, a ready-to-eat bar or ready-to-drink beverage, a capsule, a tablet, a caplet, or as a dietary gel. The most preferred dosage form is a powder beverage mix. The supplemental dietary composition may be consumed any number of times per day, e.g., one to four times per day, in order to obtain any one of the benefits set forth above.

Furthermore, the dosage form of the supplemental dietary composition may be provided in accordance with customary processing techniques for herbal and/or dietary supplements in any of the forms mentioned above. Furthermore, the supplemental dietary composition set forth in the example embodiments herein may contain any appropriate number and type of excipients, as is well known in the art.

The present invention also provides for a method for supplementing the diet of an animal, comprising administering to the animal a serving of a low carbohydrate creatine supplement comprising creatine, carbohydrate, protein and a naturally occurring free amino acid.

The present invention also provides for a method for increasing creatine accumulation in skeletal muscle of an animal comprising the steps of: administering a low carbohydrate creatine supplement comprising a serving of creatine, carbohydrate, protein and a naturally occurring free amino acid; and increasing the total muscle creatine in the skeletal muscle of an animal.

The ingestion of a high-carbohydrate creatine supplement has been shown to result in an increase in muscle creatine uptake and accumulation as compared to the ingestion of creatine alone. While not wishing to be bound by theory, it is believed that the carbohydrates increase creatine uptake by stimulating secretion of insulin. The resulting increase in plasma insulin increases the activity of a sodium-dependent muscle creatine transporter. This theory is supported by the fact that insulin augments muscle creatine accumulation in humans when present at a concentration ≧100 mU/l.

It has been unexpectedly found that the ingestion of a low carbohydrate creatine supplement comprising reduced levels of carbohydrate and protein in combination with naturally occurring free amino acids is effective to amplify creatine accumulation. The increased creatine uptake and accumulation is similar to that observed with a high-carbohydrate creatine supplement.

The low carbohydrate creatine supplement advantageously reduces the quantity of carbohydrates consumed during creatine supplementation, reducing the peak blood glucose level, and providing for a more stable blood glucose level over time. Reducing the amount of carbohydrates consumed may also help to avoid undesirable weight gain by reducing the number of empty calories.

As used herein, “total muscle creatine” refers to the total phosphocreatine and total free creatine in the skeletal muscle. Those of skill in the art will appreciate that the total muscle creatine store in a healthy, nonvegetarian subjects is, on average, about 124 mmol/kg dry mass (dm), but it can vary widely among individuals from about 100 to about 150 mmol/kg dm. The ingestion of carbohydrate free creatine (5 g creatine four times a day for 5 days) has been shown to increase total muscle creatine about 20 mmol/kg dm. The ingestion of a high-carbohydrate creatine supplement (94 g carbohydrate/5 g creatine four times per day for 5 days) has been shown to increase total muscle creatine about 35 mmol/kg dm.

As used herein, caloric content is calculated by the use of Atwater caloric conversion factors. The Atwater factors are based upon the assumptions that each gram of carbohydrate, fat, and protein in the diet will yield 4, 9, and 4 calories (kcal), respectively. Those of skill in the art will also understand that the term “empty calories” refers to foods that supply energy (calories) only, while other nutrients such as minerals, vitamins and proteins are missing or present in very low levels.

Those of skill in the art will recognize that a serving of a high-carbohydrate creatine supplement may comprise up to about 75 calories from carbohydrates, protein and naturally occurring free amino acids per gram of creatine. For example, a high-carbohydrate creatine supplement comprising about 94 g of carbohydrates per 5 g serving of creatine has about 75 cal per gram of creatine derived from carbohydrates. Commercially available creatine supplements typically comprise 30 calories per gram of creatine.

The low carbohydrate creatine supplement advantageously reduces the total number of calories needed for a serving of the supplement to increase total muscle creatine accumulation in skeletal muscle. As used herein, “a serving” refers to an amount of the low-carbohydrate creatine supplement effective in increasing creatine accumulation in skeletal muscle.

Preferably, a serving of the low carbohydrate creatine supplement comprises less than about 70 calories derived from carbohydrates, protein and naturally occurring free amino acids per gram of creatine. More preferably, a serving of the low carbohydrate creatine supplement comprises less than about 30 calories derived from carbohydrates, protein and naturally occurring free amino acids per gram of creatine. More preferably, a serving of the low carbohydrate creatine supplement comprises less than about 25 calories derived from carbohydrates, protein and naturally occurring free amino acids per gram of creatine. Most preferably, a serving of the low carbohydrate creatine supplement comprises less than about 20 calories derived from carbohydrates, protein and naturally occurring free amino acids per gram of creatine.

As used herein, “effective in increasing creatine accumulation in skeletal muscle” refers to the ability of the low-carbohydrate creatine supplement to increase total muscle creatine in skeletal muscle following ingestion of the supplement. Preferably, the increase of total muscle creatine accumulation with a serving of the low-carbohydrate creatine supplement is greater than an increase in creatine accumulation obtained with the consumption of creatine alone, that is creatine in the absence of carbohydrate, protein and naturally occurring free amino acids.

In a preferred embodiment, the low carbohydrate creatine supplement increases total muscle creatine greater than about 20 mmol/kg dm when administered as four servings per day for 5 days. In a more preferred embodiment, the low carbohydrate creatine supplement increases total muscle creatine about 24 mmol/kg dm when administered as four servings per day for five days. In an even more preferred embodiment, the low carbohydrate creatine supplement increases total muscle creatine about 28 mmol/kg dm when administered as four servings per day for five days. Most preferably, the low carbohydrate creatine supplement increases total muscle creatine about 33 mmol/kg dm when administered as four servings per day for 5 days.

Those of skill in the art will appreciate that the increase of total muscle creatine with the supplement refers to an average increase of total muscle creatine over a statically large population and that the increase will vary between individuals. In particular individuals with some degree of insulin resistance may have significantly lower creatine increase than the average.

Clinical determination of creatine accumulation in skeletal muscle following ingestion of the low carbohydrate creatine supplement may be measured by various methods well known to those of skill in the art. For example, creatine accumulation in skeletal muscle can be measured directly by muscle biopsy.

Direct measurement of creatine accumulation in muscle may involve taking biopsy samples from a subject. Biopsy samples are preferably frozen in liquid nitrogen, freeze-dried, and stored at −80° C. for subsequent metabolite analysis. Typically, fat is removed from the freeze dried sample by extraction with petroleum ether, muscle samples dissected free from visible blood and connective tissue and then powdered. Neutralized perchloric acid extracts may then be prepared for the spectrophotometric determination of phosphocreatine and creatine. Total muscle creatine concentration may be calculated by summing phosphocreatine and free creatine concentrations.

Creatine accumulation in skeletal muscle following ingestion of the low carbohydrate creatine supplement can be estimated indirectly. Subjects ingesting creatine in combination with the low carbohydrate creatine supplement of the invention have plasma creatine concentration and urinary creatine excretion substantially decreased when compared with creatine ingestion alone, indicating that whole body creatine retention was increased.

Measurement of creatine levels in the plasma preferably involves removing venous blood from the dorsal surface of a heated hand immediately before and 20, 40, and 60 min after the ingestion of a supplement. In addition, urine may be collected before and on the day of ingestion of a supplement. Plasma and urine creatine may be measured using high performance liquid chromatography and serum insulin was measured using a radioimmunoassay technique, an example of which is described in U.S. Pat. No. 5,968,900, which is fully incorporated herein by reference.

The present invention may provide a low carbohydrate creatine supplement comprising; creatine, carbohydrate, protein and a naturally occurring free amino acid wherein a serving of the supplement is effective in increasing creatine accumulation in skeletal muscle.

As used herein, “creatine” refers to the chemical compound N-methyl-N-guanyl glycine, CAS Registry No. 57-00-1, also known as, (α-methyl guanido) acetic acid, N-(aminoiminomethyl)-N-glycine, and methylglycocyamine, and Methylguanidoacetic acid, and N-Methyl-N-guanylglycine. As used herein, “creatine” also includes derivatives of creatine such as esters, and amides, as well as other derivatives, including derivatives that become active upon metabolism. The structure of creatine is shown below.

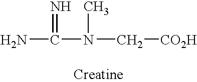

Creatine and creatine derivatives are widely available from a number of commercial sources. Commercially available creatine derivatives include creatine phosphate, creatine citrate, magnesium creatine, alkaline creatine, creatine pyruvate, creatine hydrates, and tricreatine malate. Glycocyamine, and in vivo precursor of creatine, are also commercially available and suitable in the practice of the present invention.

As used herein, a serving of the supplement comprises from about 0.5 g to about 30 g of creatine. More preferably, a serving of the supplement comprises from about 2 g to about 20 g of creatine. In various example embodiments, a serving of the supplement comprises about 5 g or about 10 g of creatine.

As used herein, “carbohydrate” preferably refers to food carbohydrates such as simple carbohydrates and polysaccharides and combinations thereof; as well as derivatives thereof such as esters, and amides, as well as other derivatives, including derivatives that become active upon metabolism.

Simple carbohydrates may refer to glucose, maltose, sucrose, galactose and lactose or combinations thereof. Advantageously, the simple carbohydrate is glucose. Polysaccharides may refer to maltodextrin, starch and glycogen or combinations thereof. Advantageously, the simple polysaccharides refers to maltodextrin.

The carbohydrate may be a combination of a simple carbohydrate and a polysaccharide. When the carbohydrate refers to a combination of a simple carbohydrate and a polysaccharide, a weight ratio of simple carbohydrate to polysaccharide may be from about 1 to 2 parts to about 2 to 1. Preferably, the weight ratio is about 1 to 1.

In various embodiments, a serving of the low carbohydrate creatine supplement comprises less than about 7.4 g of carbohydrates per gram of creatine. More preferably, a serving of the low carbohydrate creatine supplement comprises less than about 6.0 g of carbohydrates per gram of creatine. More preferably, a serving of the low carbohydrate creatine supplement comprises less than about 4.0 g of carbohydrates per gram of creatine. Most preferably, a serving of the low carbohydrate creatine supplement comprises no more than about 3.0 g of carbohydrates per gram of creatine.

As used herein, “protein” may refer to food proteins but also includes dipeptides, tripeptides, polypeptides as well as derivatives thereof such as esters, and amides, as well as other derivatives, including derivatives that become active upon metabolism.

The protein portion of the supplement may be a dairy protein or non-dairy protein. A preferred non-dairy protein is soy protein. Dairy proteins may include high quality milk proteins and whey proteins. High quality milk proteins include isolates and concentrates of milk proteins. High quality milk proteins are predominantly caseins. Whey proteins include whey isolates and whey concentrates. Whey isolates include whey hydrolysate. Advantageously, the protein is a dairy protein selected from a group consisting of casein and whey protein, e.g., whey hydrolysate.

A serving of the supplement may include from about 0.1 g to about 9.0 g of protein per gram of protein. More preferably, a serving of the supplement comprises from about 0.2 g to about 7.5 g of protein per gram of creatine. Most preferably, a serving of the supplement comprises from about 1.0 g to about 6.0 g of protein per gram of creatine.

As used herein, a “naturally occurring free amino acid” refers to amino acids used for protein synthesis in mammals including derivatives of amino acids such as esters, and amides, as well as other derivatives, including derivatives that become active upon metabolism. The naturally occurring free amino acids may be selected from the group consisting of: glycine, alanine, valine, leucine, isoleucine, serine, threonine, cysteine, methionine, aspartic acid, asparagine, glutamic acid, glutamine, arginine, lysine, histidine, phenylalanine, tyrosine, tryptophan and proline as well as derivatives thereof.

The low carbohydrate creatine supplement may comprise at least one naturally occurring free amino acid. More preferably, the supplement comprises at least one naturally occurring free amino acid selected from the group consisting of L-Leucine and L-Phenylalanine. Most preferably the supplement comprises both L-Leucine and L-Phenylalanine.

Preferably, a serving of the supplement comprises from about 0.1 g to about 9.0 g of a naturally occurring free amino acid per gram of creatine. More preferably, a serving of the supplement comprises from about 0.2 g to about 7.5 g of a naturally occurring free amino acid per gram of creatine. More preferably, a serving of the supplement comprises from about 1.0 g to about 6.0 g of a naturally occurring free amino acid per gram of creatine. Most preferably, a serving of the supplement comprises about 1.44 g L-Leucine, and about 1.44 g L-Phenylalanine per gram of creatine.

Additional ingredients, which increase creatine accumulation in skeletal muscle, may advantageously be added to the low carbohydrate creatine supplement to further reduce the empty calories. Optionally additional ingredients may be selected from the group consisting of alpha lipoic acid, hydroxy-isoleucine, a chromium chelate and L-taurine as well as including derivatives thereof such as esters, and amides, as well as other derivatives, including derivatives that become active upon metabolism.

Alpha lipoic acid is an insulin modulator and an antioxidant that serves as protection against oxidative injury in non-neuronal and neuronal tissue. A serving of the low carbohydrate creatine supplement may include from about 100 mg to about 1 mg of alpha lipoic acid per gram of creatine. More preferably a serving of the low carbohydrate creatine supplement includes from about 50 mg to about 5 mg of alpha lipoic acid per gram of creatine. Even more preferably, a serving of the low carbohydrate creatine supplement includes from about 30 mg to about 10 mg of alpha lipoic acid per gram of creatine. In the most preferred embodiment, a serving of the low carbohydrate creatine supplement includes about 20 mg of alpha lipoic acid per gram of creatine.

L-Taurine is an amino acid which is not involved in the synthesis of proteins in animals and is the end product of L-cysteine metabolism. L-Taurine is the principle free intracellular amino acid found in human tissue. L-taurine also is antioxidant, and has been shown to improve insulin sensitivity. A serving of the low carbohydrate creatine supplement preferably may include from about 1.0 g to about 10 mg of L-taurine per gram of creatine. More preferably a serving of the low carbohydrate creatine supplement includes from about 500 mg to about 20 mg of L-taurine per gram of creatine. In the most preferred embodiment, a serving of the low carbohydrate creatine supplement includes about 200 mg of L-taurine per gram of creatine.

Chromium has been shown to improve insulin sensitivity and glucose disposal. Chromium is supplied as a chromium chelate. Preferred chromiun chelate include chromium picolinate and chromium nicotinate. A serving of the low carbohydrate creatine supplement may supply from about 100 mcg to about 5 mcg of chromium per gram of creatine. More preferably a serving of the low carbohydrate creatine supplement supplies from about 50 mcg to about 10 mcg of chromium per gram of creatine. In the most preferred embodiment, a serving of the low carbohydrate creatine supplement includes about 30 mcg of chromium per gram of creatine.

4-Hydroxyisoleucine is an amino acid that occurs naturally in fenugreek seeds, but does not occur naturally in mammalian muscle tissue. 4-Hydroxyisoleucine has been shown to improve insulin sensitivity. See U.S. Pat. No. 5,470,879, hereby incorporated by reference in its entirety. A serving of the low carbohydrate creatine supplement preferably includes from about 100 mg to about 10 g of 4-Hydroxyisoleucine per gram of creatine. More preferably a serving of the low carbohydrate creatine supplement includes from about 500 mg to about 5 g of 4-Hydroxyisoleucine per gram of creatine. In the most preferred embodiment, a serving of the low carbohydrate creatine supplement includes about 2 g of 4-Hydroxyisoleucine per gram of creatine.

The supplement of the invention preferably comprises less than 7 grams of fat per serving. More preferably the supplement comprises less than 5 gram of fat per serving. Most preferably the supplement comprises less than 3 grams of fat per serving.

Those of skill in the art will appreciate that the supplement may comprise small amounts of free fatty acids either for health benefits or for packaging.

When the supplement is supplied as a dry powder, a serving of the dry powdered supplement may be mixed with 8 ounces of water or a liquid sports drink for consumption by an person. Following consumption of the supplement, 8-16 ounces of water or an athletic drink may be consumed by a person.

When the supplement is provided as other dosage forms, such as a capsule, or as a ready-to-eat bar product, the supplement may be consumed by a person with 8-16 ounces of water or an athletic drink.

In one embodiment a serving of the low carbohydrate creatine supplement is consumed by an athlete 1-4 times per day for five days. More preferably, a serving of the supplement is administered 2 times a day for five days. In an alternative embodiment a serving of the supplement is administered 2 times a day 12 hours apart for five days. More preferably, a serving of the supplement is administered 2 times a day, once in the morning and again after a workout for five days. In a further alternative embodiment the supplement is taken every day for an indefinite period of time immediately after a workout. In an alternative embodiment the supplement is taken every day for an indefinite period of in the morning on an empty stomach.

In addition, the present invention relates to a method of manufacturing a supplemental dietary composition that may activate the protein synthesis machinery and deactivate catabolic processes within skeletal muscle by regulating molecular signals to control anabolic and anti-catabolic activity in skeletal muscle, and in doing so, that may stimulate muscle growth, increase muscle mass, increase weight gain, decrease muscle catabolism and associated muscle and weight loss, increase performance, improve body composition, treat muscle wasting or degenerative disease, suppress the effects of sarcopenia in the aging population and/or provide a beneficial effect by influencing the genetic control system for global protein synthesis. For example, the method of manufacturing a supplemental dietary composition may include the step of mixing L-Leucine, including salts or derivatives thereof, L-phenylalanine, including salts or derivatives thereof, and/or creatine, including salts or derivatives therof, with one or more of sources of dietary protein and/or carbohydrates. Any of the various ingredients described in Examples 1 through 10 may also be added. The method of manufacturing the supplemental dietary composition may also include the step of checking for uniformity/homogeneity. In addition, the method of manufacturing the supplemental dietary composition, may include the step of aliquoting the mixture into a serving for, e.g., compression into a caplet.

The present invention also provides for a method of manufacturing a low carbohydrate creatine supplement comprising the following steps: premixing microcrystalline cellulose with the following ingredients to the premix creatine, dextrose, high quality milk proteins, L-Phenylalanine, L-Leucine, and microcrystalline cellulose; adding magnesium stearate and silica which had been pre-sifted; blending and mixing for 30 minutes; checking for uniformity and/or homogeneity and then aliquoting into a serving.

By activating signal transduction pathways (both mTOR dependent and independent) in combination with the benefits of creatine, the present invention provides a novel way to ensure the anabolic machinery is operating in a favorable manner to promote an anabolic environment within muscles to help optimize protein synthesis. The present invention may provide an advantage over conventional products that purport to stimulate protein synthesis but lack, or include in insufficient quantities, the correct signaling promoting nutritive agents, specifically leucine (the most potent of the branch chain amino acids which induces anabolism in muscle) and directly and/or indirectly phenylalanine to ensure proper translation initiation for muscle building and to decrease or inhibit catabolism.

Although the following examples illustrate the practice of the present invention in some of its embodiments, the examples should not be construed as limiting the scope of the invention. Other embodiments will be apparent to one skilled in the art from consideration of the specification and examples.

### EXAMPLE 1

A creatine supplement comprising the following ingredients per serving is prepared as a dry powder for consumption by an individual, e.g., athlete.

| INGREDIENT | GRAMS PER SERVING |
|---|---|
| Maltodextrin | 6.500 |
| Creatine monohydrate | 5.000 |
| Whey protein Isolate | 1.000 |
| Taurine | 0.500 |
| Citric acid | 0.431 |
| Flavoring | 0.426 |
| Alpha lipoic acid | 0.250 |
| Ascorbic acid | 0.214 |
| dipotassium phosphate | 0.150 |
| magnesium phosphate | 0.150 |
| Tricreatine malate | 0.150 |
| Dicreatine malate | 0.150 |
| Creatine ethyl ester | 0.150 |
| L-Leucine | 0.150 |
| L-Phenylalanine | 0.150 |
| Disodium phosphate | 0.150 |
| Betaine | 0.132 |
| Acesulfame Potassium | 0.114 |
| Sucralose | 0.075 |
| Coloring | 0.020 |
| fenugreek extract | 0.010 |
| D-pinitol | 0.002 |
| Chromium polynicotinate | 0.001 |
| Total serving size: | 15.725 |

### EXAMPLE 2

A 15.7 g of the dry powder of the low calorie creatine supplement is mixed with 8 ounces of water and consumed by an athlete 4 times per day for five days. After five days of consuming the low calorie creatine supplement athlete's total muscle creatine has increased 33 mmol/kg dm.

### EXAMPLE 3

A creatine supplement comprising the following ingredients per serving is prepared as a dry powder for consumption by an individual, e.g., athlete.

| INGREDIENT | GRAMS PER SERVING |
|---|---|
| Maltodextrin | 6.500 |
| Creatine monohydrate | 5.000 |
| Whey protein Isolate | 1.000 |
| Taurine | 0.500 |
| Citric acid | 0.431 |
| Flavoring | 0.426 |
| Alpha lipoic acid | 0.250 |
| dipotassium phosphate | 0.150 |
| magnesium phosphate | 0.150 |
| Tricreatine malate | 0.150 |
| Dicreatine malate | 0.150 |
| L-Leucine | 0.150 |
| L-Phenylalanine | 0.150 |
| Disodium phosphate | 0.150 |
| Betaine | 0.132 |
| Acesulfame Potassium | 0.114 |
| Sucralose | 0.075 |
| Coloring | 0.020 |
| Total serving size: | 15.498 |

### EXAMPLE 4

A 15.5 g of the dry powder of the low calorie creatine supplement is mixed with 8 ounces of water and consumed by an athlete 4 times per day for five days. After five days of consuming the low calorie creatine supplement athlete's total muscle creatine has increased 33 mmol/kg dm.

### EXAMPLE 5

A creatine supplement comprising the following ingredients per serving is prepared as a dry powder for consumption by an individual, e.g., athlete.

| Ingredients | g/serving |
|---|---|
| Dextrose | 1.0-95.0 |
| Maltodextrin | 1.0-95.0 |
| Partly Hydrolyzed Whey Protein | 14.000 |
| L-Leucine (as one or more of l- | 7.200 |
| leucine, leucine AKG, leucine ethyl | |
| ester, n-acetyl leucine, and nor- | |
| leucine) | |
| L-Phenylalanine | 7.200 |
| Creatine monohydrate | 10.000 |
| Alpha-lipoic acid | 0.001-0.300 |
| Xanthan Gum | 0.112 |
| Flavoring | 2.000 |
| Coloring | 0.090 |

### EXAMPLE 6

An 97.6 g of the dry powder of the low calorie creatine supplement is mixed with 8 ounces of water and consumed by an athlete 4 times per day for five days. After five days of consuming the low calorie creatine supplement, athlete's total muscle creatine has increased 33 mmol/kg dm.

### EXAMPLE 7

A creatine supplement comprising the following ingredients per serving is prepared as a dry powder for consumption by an individual, e.g., athlete.

| | Dietary |
|---|---|
| | Ingredient per |
| Dietary Ingredient Name | serving (g) |
| Dextrose 99 DE | 28.5000 |
| Maltodextrin | 28.5000 |
| WPC-80, part hydrolyzed | 14.0000 |
| L-Leucine (as one or more of l-leucine, | 7.2000 |
| leucine AKG, leucine ethyl ester, n- | |
| acetyl leucine, and nor-leucine) | |
| L-Phenylalanine | 7.2000 |
| Creatine monohydrate, fine grind | 5.0000 |
| Alpha-lipoic acid | 0.001-0.300 |
| Other Ingredients | g/serving |
| Bitter blocker flavor | 0.4600 |
| Citric acid, fine gran | 0.3490 |
| Banana flavor, N&A | 0.2760 |
| Potassium citate, 36% | 0.2300 |
| Sucralose | 0.1380 |
| Pineapple flavor, N&A | 0.0460 |
| FD&C Yellow #5 | 0.0030 |
| | 1.5020 |

### EXAMPLE 8

A creatine supplement comprising the following ingredients per serving is prepared as a dry powder for consumption by an individual, e.g., athlete.

| | Dietary |
|---|---|
| | Ingredient |
| | per serving |
| Dietary Ingredient Name | (g) |
| Dextrose 99 DE | 28.5000 |
| Maltodextrin | 28.5000 |
| WPC-80, part hydrolyzed | 14.0000 |
| L-Leucine | 7.2000 |
| L-Phenylalanine | 7.2000 |
| Creatine monohydrate, fine grind | 5.0000 |
| Alpha-lipoic acid | 0.100 |
| | 90.5000 |
| Other Ingredients | g/serving |
| Bitter blocker flavor | 0.4600 |
| Citric acid, fine gran | 0.3490 |
| Banana flavor, N&A | 0.2760 |
| Potassium citate, 36% | 0.2300 |
| Sucralose | 0.1380 |
| Pineapple flavor, N&A | 0.0460 |
| FD&C Yellow #5 | 0.0030 |
| Weights (grams) | 1.5020 |
| Total Weight | 92.0020 |

### EXAMPLE 9

A creatine supplement comprising the following ingredients per serving is prepared as a dry powder for consumption by an individual, e.g., athlete.

| | Dietary |
|---|---|
| | Ingredient |
| | per serving |
| Dietary Ingredient Name | (g) |
| Dextrose 99 DE | 28.5000 |
| Maltodextrin | 28.5000 |
| WPC-80, part hydrolyzed | 14.0000 |
| L-Leucine | 7.2000 |
| L-Phenylalanine | 7.2000 |
| Creatine monohydrate, fine grind | 5.0000 |
| Alpha-lipoic acid | 0.200 |
| | 90.5000 |
| Other Ingredients | g/serving |
| Bitter blocker flavor | 0.4600 |
| Citric acid, fine gran | 0.3490 |
| Banana flavor, N&A | 0.2760 |
| Potassium citate, 36% | 0.2300 |
| Sucralose | 0.1380 |
| Pineapple flavor, N&A | 0.0460 |
| FD&C Yellow #5 | 0.0030 |
| Weights (grams) | 1.5020 |
| Total Weight | 92.1020 |

### EXAMPLE 10

A creatine supplement comprising the following ingredients per serving is prepared as a dry powder for consumption by an individual, e.g., athlete.

| | Dietary |
|---|---|
| | Ingredient |
| | per serving |
| Dietary Ingredient Name | (g) |
| Maltodextrin | 57.0000 |
| WPC-80, part hydrolyzed | 14.0000 |
| L-Leucine | 7.2000 |
| L-Phenylalanine | 7.2000 |
| Creatine monohydrate, fine grind | 5.0000 |
| | 90.4000 |
| Other Ingredients | g/serving |
| Bitter blocker flavor | 0.4600 |
| Citric acid, fine gran | 0.3490 |
| Banana flavor, N&A | 0.2760 |
| Potassium citate, 36% | 0.2300 |
| Sucralose | 0.1380 |
| Pineapple flavor, N&A | 0.0460 |
| FD&C Yellow #5 | 0.0030 |
| | 1.5020 |
| | 91.9020 |

### EXAMPLE 11

### Manufacturing the Low Carbohydrate Creatine Supplement

1. PREMIX: Chromium Chelate and microcrystalline cellulose (MCC) 102 is premixed separately for 10 minutes.

2. Add the following ingredients are added to the premix from step 1, creatine monohydrate, dextrose, high quality milk proteins, L-Phenylalanine, L-Leucine, and microcrystalline cellulose and sifted through a mesh #10 The ingredients are then added into the mixer and mixed for 60 minutes.

3. Magnesium Stearate and silica are then pre-sifted through mesh #30 and added to the mixture from step 2 and blended and mixed for 30 minutes.

4. The product is checked for uniformity/homogeneity.

5. The product is then aliquoted into dry batches comprising 100 servings.

### EXAMPLE 12

### Optimization of Creatine Retention in Man

Aim: The aim of this study was to identify a supplement that would optimize the augmentation of Cr retention after its supplementation, by increasing the insulinotropic effect, whilst consuming a lower carbohydrate load.

Methods:

Study design: Randomized, double-blind, placebo controlled, cross-over design.

Ethical approval: This study was approved by the University of Nottingham Medical School Research Ethics Committee.

Volunteers: 7 healthy male volunteers. All volunteers were eligible to participate after satisfactory results from the medical screening.

Protocol: The volunteers were required to attend to the lab for 3 trials. Each consisted of an afternoon arm, and a morning arm. Each arm lasted for approximately 4 hours. The volunteers were asked to relax on a bed. A baseline blood sample was taken. Each solution was administered via a nasogastric tube (mean time of administration was approximately 7 minutes). Half-way through the administration, the stop clock was started. After the three hour protocol, a second solution was administered. The third solution was administered in the following morning arm of the trial. Each trial was separated by at least 12 days.

Solution Mixtures:

Solution A: 5 g creatine (Cr)+water (C)

Solution B: 5 g Cr+˜95 g dextrose (CHO).

Solution C: 5 g Cr+57 g dextrose+28 g protein/amino acids (50/50) (PAC).

Each solution was administered via a nasogastric tube three times over 24 hours. A total amount of 15 g or Cr was administered.

Blood sampling: Blood samples were collected for three hours after administration of the solution. Eleven blood samples obtained (including baseline sample). For the first hour after administration of the solution, a blood sample was obtained at 15 minute intervals. During the second and third hour of sampling, the intervals were increased to 20 minutes. Approximately 3 ml of blood was transferred to a lithium heparin containing tube and a further 3 ml were allowed to clot for plasma Cr and creatinine (Crn), and serum insulin analysis respectively.

Urinary Creatine Content:

Three 24 h hour urinary collections were obtained from each volunteer for each arm of the study. The first collection (baseline) was completed prior to administration of the solution. The second collection (0-24 h) was initiated immediately after the administration of the first solution, until 24 h post administration. The third collection (24-48 h) followed that of the 0-24 h collection. The volume of the urine excreted was recorded and a 5 ml sample was frozen at −20° C. until analysis. The samples were analyzed for Cr and Crn.

To calculate the total creatine (TCr) the Cr and Crn were added together. The TCr increase was calculated by subtracting the baseline TCr from the 0-24 h excretion and/or the 24-48 h excretion. The 0-48 h content was calculated by adding the 0-24 h TCr with the 24-48 h TCr increase in excretion.

Statistical analysis: A two-way repeated measure ANOVA statistical test was used. Significance was set at p<0.05. When a significant difference was observed, Fisher's post hoc analysis was performed in order to locate the difference. All individual results are included in the appendix.

Results:

All figures are plotted using the means. The error bars represent the standard error of the means.

TABLE 1
Individual characteristics.

| Subject | Age (y) | Height (cm) | Weight (kg) | BMI |
|---|---|---|---|---|
| S1 | 24 | 183.5 | 72.3 | 21 |
| S2 | 22 | 183.5 | 68.8 | 20 |
| S6 | 26 | 184 | 91.4 | 27 |
| S7 | 29 | 181 | 74.1 | 23 |
| S13 | 25 | 178.5 | 101.6 | 32 |
| S15 | 23 | 183 | 93.2 | 28 |
| S16 | 24 | 187.5 | 87.7 | 25 |
| Mean | 25 | 183 | 84 | 25 |
| SE | 1 | 1 | 5 | 2 |

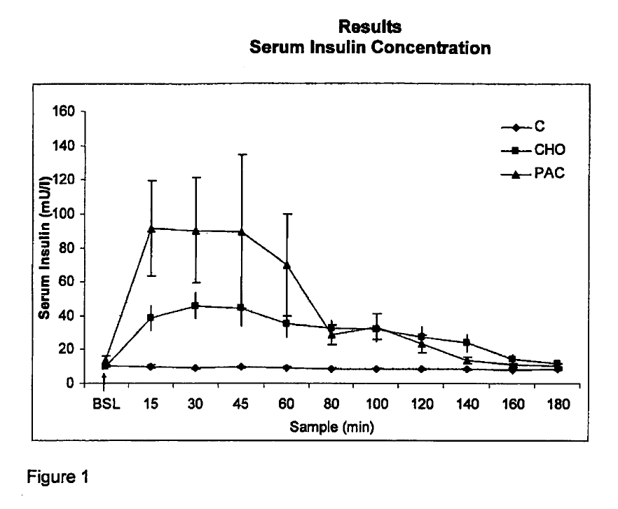

Referring to the accompanying figures, FIG. 1 is a diagram that illustrates serum insulin concentration (mU/I) following the first oral challenge with C, CHO, and PAC. The insulin concentration after administration of C was significantly lower when compared to CHO from 15-160 min, and PAC from 15-140 min. The concentration after CHO was significantly lower when compared to PAC at 15 minutes.

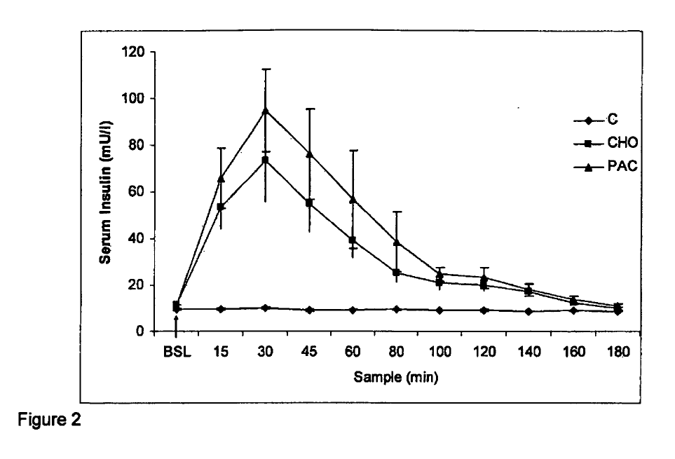

FIG. 2 is a diagram that illustrates serum insulin concentration (mU/l) following the third oral challenge with C, CHO, and PAC. The insulin concentration after administration of C was significantly lower when compared to CHO and PAC from 15-160 min.

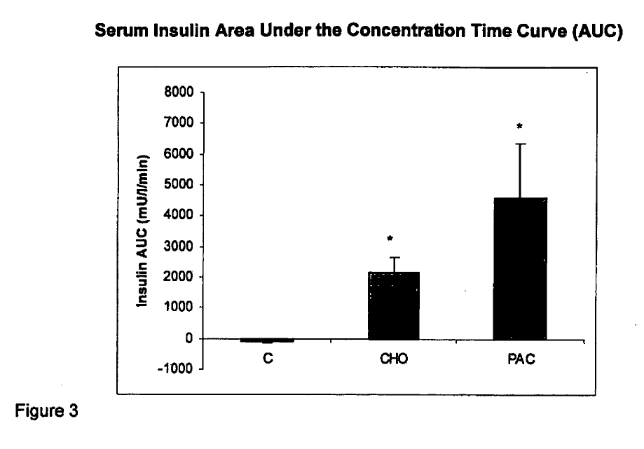

FIG. 3 is a diagram that illustrates serum insulin area under the concentration time curve for 80 min following the first oral challenge with C, CHO, and PAC. Insulin AUC is significantly lower (*) after administration of C when compared to CHO and PAC (p=0.02).

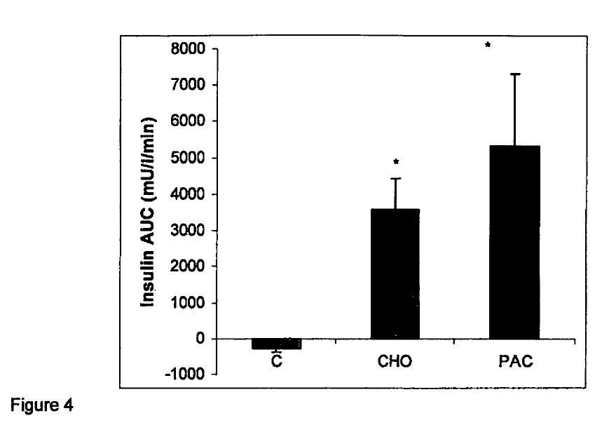

FIG. 4 is a diagram that illustrates serum insulin area under the concentration time curve for 180 min following the first oral challenge with C, CHO, and PAC. Insulin AUC is significantly lower (*) after administration of C when compared to CHO and PAC (p=0.015).

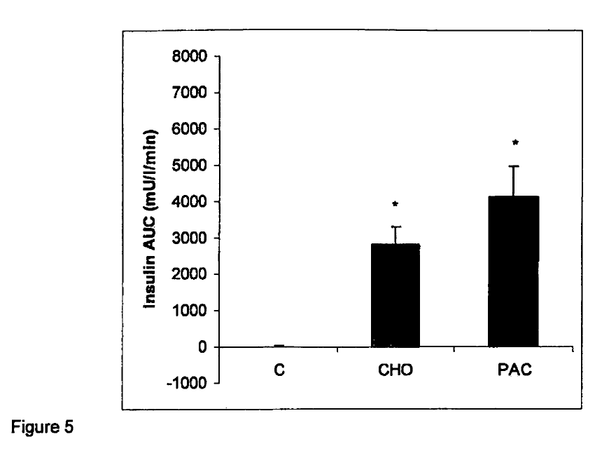

FIG. 5 is a diagram that illustrates serum insulin area under the concentration time curve for 80 min following the third oral challenge with C, CHO and PAC. Insulin AUC is significantly lower (*) after administration of C when compared to CHO and PAC (p<0.001).

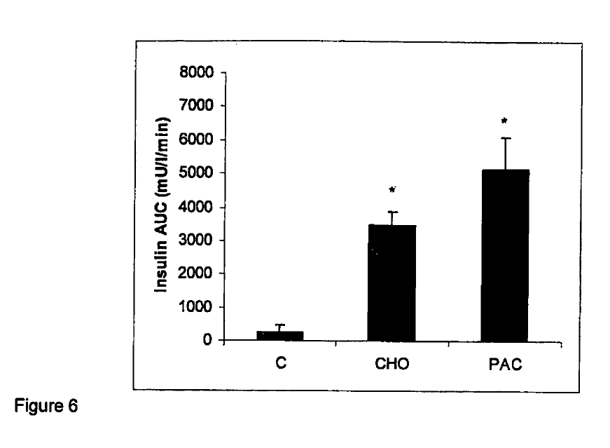

FIG. 6 is a diagram that illustrates serum insulin area under the concentration time curve for 180 min following the first oral challenge with C, CHO and PAC. Insulin AUC is significantly lower (*) after administration of C when compared to CHO and PAC (p<0.001).

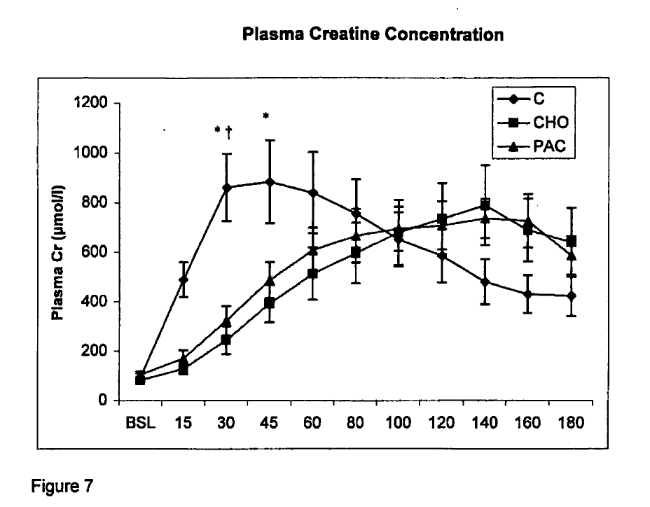

FIG. 7 is a diagram that illustrates plasma creatine concentration (μmol/l) following the first oral challenge with C, CHO and PAC. The plasma creatine concentration was significantly higher (p<0.05) after administration of C from 15-60 min compared to CHO (*) and 15-30 min compared to PAC (†).

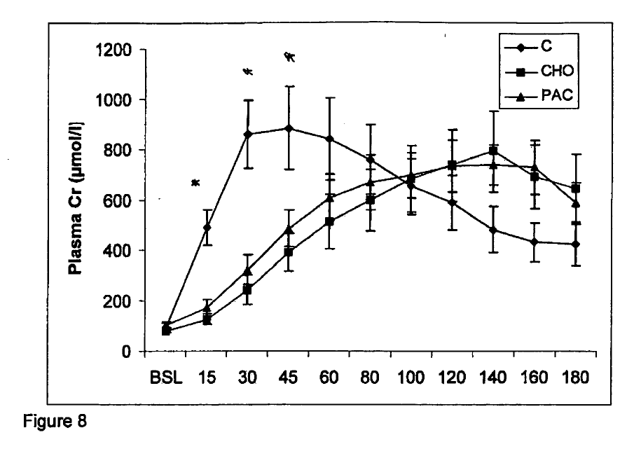

FIG. 8 is a diagram that illustrates plasma creatine concentration (μmol/l) following the third oral challenge with C, CHO and PAC. The plasma creatine concentration was significantly higher (p<0.05) after administration of C at 15-45 min compared to CHO and PAC (*).

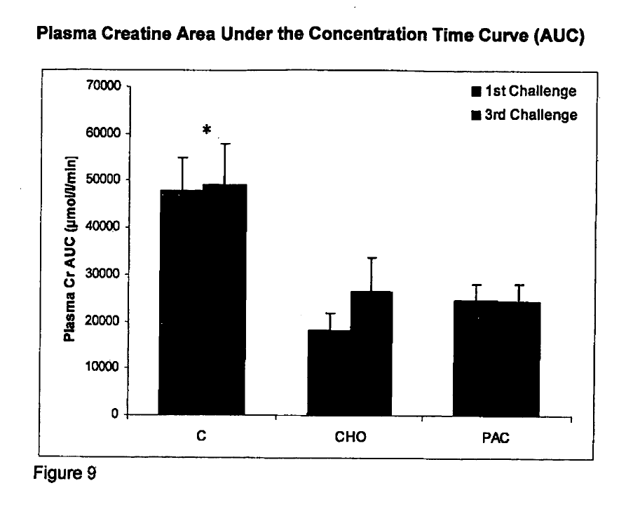

FIG. 9 is a diagram that illustrates plasma creatine AUC (μmol/l/min) 80 min following the first and third oral challenge with C, CHO and PAC. The AUC is significantly greater (*) after administration of C compared to CHO and PAC after both, first and third oral challenge (p<0.05).

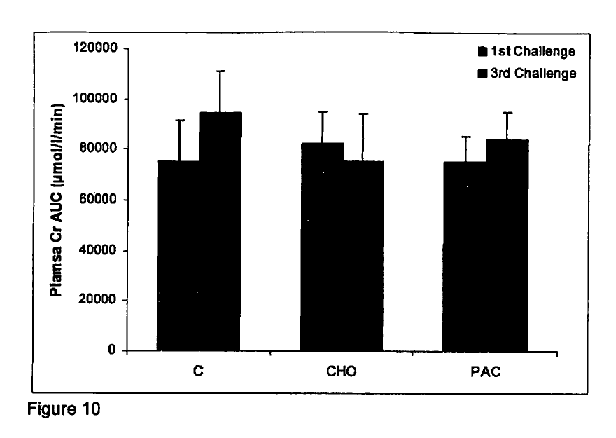

FIG. 10 is a diagram that illustrates plasma creatine AUC (μmol/l/min) 180 min following the first and third oral challenge with C, CHO and PAC. No significant differences were found between the treatments.

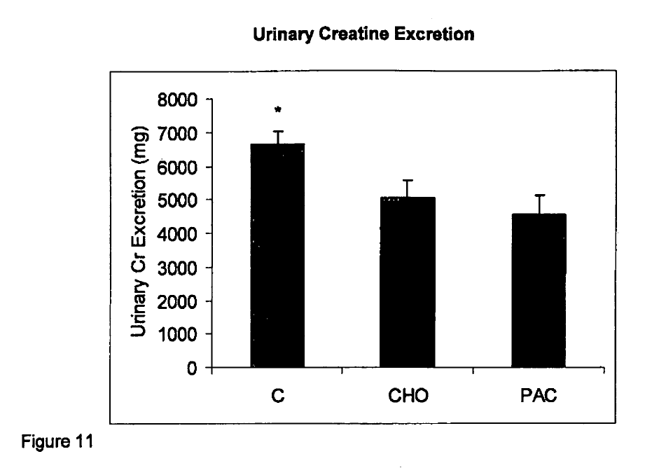

FIG. 11 is a diagram that illustrates urinary creatine excretion (mg) 0-24 h. Urinary creatine content after C (*) was significantly greater when compared to CHO, and PAC (p<0.05).

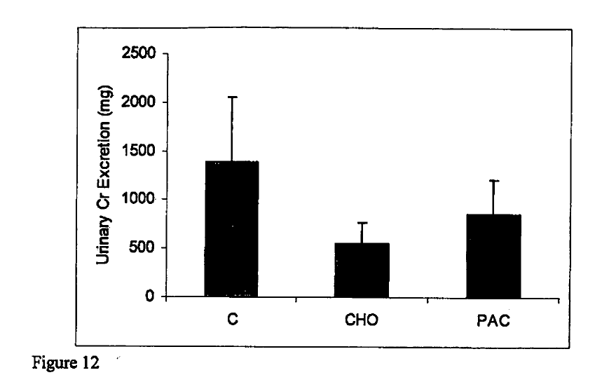

FIG. 12 is a diagram that illustrates urinary creatine excretion (mg) 24-48 h following administration. There was no significant difference between the trials.

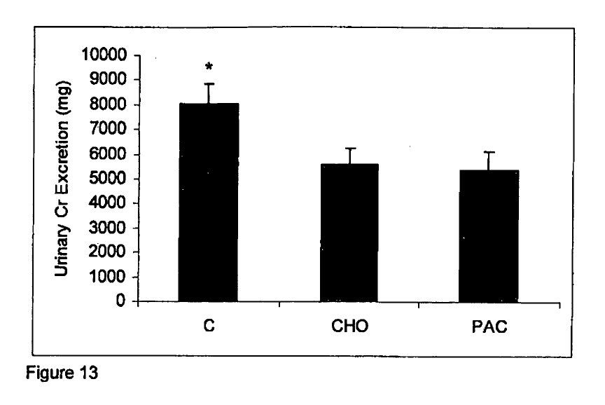

FIG. 13 is a diagram that illustrates urinary creatine excretion (mg) 0-48 h following supplementation. Urinary creatine content after solution C (*) was significantly greater when compared to CHO and PAC (p<0.05).

FIG. 14 is a diagram that illustrates the signaling events involved in the stimulation of translation initiation.

### Appendix: Serum Insulin Concentration

TABLE 2
Individual serum insulin concentration (mU/l) after the first oral
challenge with C.

| | Subject | | | | | | | | |
|---|---|---|---|---|---|---|---|---|---|
| Timepoint (min) | S1 | S2 | S6 | S7 | S13 | S15 | S16 | Mean | SEM |
| 0 | 8.94 | 11.62 | 13.15 | 9.47 | 9.58 | 10.38 | 10.52 | 10.52 | 0.55 |
| 15 | 9.60 | 7.45 | 14.52 | 8.90 | 8.01 | 9.79 | 11.43 | 9.96 | 0.90 |
| 30 | 8.04 | 7.56 | 8.56 | 8.74 | 9.20 | 9.50 | 11.18 | 8.97 | 0.44 |
| 45 | 9.02 | 7.85 | 9.58 | 10.16 | 9.61 | 9.34 | 11.85 | 9.63 | 0.46 |
| 60 | 9.17 | 7.54 | 9.74 | 8.51 | 9.02 | 10.16 | 10.11 | 9.18 | 0.35 |
| 80 | 7.63 | 6.88 | 10.70 | 8.92 | 8.60 | 9.55 | 8.94 | 8.74 | 0.47 |
| 100 | 7.56 | 8.32 | 10.77 | 7.52 | 8.86 | 9.85 | 8.53 | 8.77 | 0.45 |
| 120 | 6.98 | 7.49 | 8.32 | 8.27 | 9.23 | 9.89 | 8.46 | 8.38 | 0.37 |
| 140 | 8.11 | 7.14 | 7.82 | 7.10 | 8.38 | 9.55 | 9.28 | 8.20 | 0.36 |
| 160 | 7.52 | 7.30 | 7.85 | 6.53 | 8.58 | 9.69 | 8.26 | 7.96 | 0.38 |
| 180 | 7.93 | 7.21 | 7.94 | 6.65 | 9.19 | 10.04 | 8.56 | 8.22 | 0.44 |

TABLE 3
Individual serum insulin concentration (mU/l) following the third oral
challenge with C.

| | Subject | | | | | | | | |
|---|---|---|---|---|---|---|---|---|---|
| Timepoint (min) | S1 | S2 | S6 | S7 | S13 | S15 | S16 | Mean | SEM |
| 0 | 8.91 | 8.31 | 10.38 | 8.91 | 9.64 | 10.31 | 8.65 | 9.30 | 0.31 |
| 15 | 8.94 | 7.78 | 10.23 | 8.94 | 9.84 | 10.29 | 11.08 | 9.59 | 0.42 |
| 30 | 9.44 | 7.90 | 10.92 | 9.44 | 10.89 | 9.80 | 11.61 | 10.00 | 0.47 |
| 45 | 8.87 | 7.24 | 9.56 | 8.87 | 9.07 | 10.07 | 9.32 | 9.00 | 0.34 |
| 60 | 8.92 | 7.99 | 10.03 | 8.92 | 8.61 | 9.69 | 10.81 | 9.28 | 0.36 |
| 80 | 9.06 | 8.54 | 9.79 | 9.06 | 9.45 | 10.22 | 8.93 | 9.29 | 0.21 |
| 100 | 8.54 | 8.56 | 9.59 | 8.54 | 9.34 | 9.59 | 9.38 | 9.08 | 0.19 |
| 120 | 8.76 | 8.20 | 10.07 | 8.76 | 8.48 | 10.27 | 9.10 | 9.09 | 0.30 |
| 140 | 8.29 | 8.44 | 8.57 | 8.29 | 8.55 | 8.56 | 9.20 | 8.56 | 0.12 |
| 160 | 8.69 | 8.34 | 8.66 | 8.69 | 10.01 | 9.02 | 9.73 | 9.02 | 0.23 |
| 180 | 8.58 | 8.61 | 8.58 | 8.58 | 8.56 | 9.43 | 9.04 | 8.77 | 0.13 |

TABLE 4
Individual serum insulin concentration (mU/l) after the first oral
challenge with CHO.

| | Subject | | | | | | | | |
|---|---|---|---|---|---|---|---|---|---|
| Timepoint (min) | S1 | S2 | S6 | S7 | S13 | S15 | S16 | Mean | SEM |
| 0 | 9.81 | 13.90 | 8.53 | 7.22 | 9.03 | 10.85 | 9.61 | 9.85 | 0.80 |
| 15 | 29.64 | 52.23 | 21.22 | 24.25 | 22.51 | 67.53 | 52.70 | 38.58 | 7.02 |
| 30 | 28.10 | 55.62 | 31.46 | 28.37 | 41.40 | 52.77 | 84.45 | 46.02 | 7.69 |
| 45 | 26.49 | 36.95 | 40.25 | 39.70 | 37.35 | 26.28 | 102.77 | 44.26 | 10.00 |
| 60 | 15.02 | 24.97 | 42.03 | 29.81 | 23.79 | 30.92 | 81.87 | 35.49 | 8.33 |
| 80 | 17.85 | 20.88 | 35.23 | 42.07 | 24.00 | 30.14 | 56.45 | 32.37 | 5.12 |
| 100 | 29.98 | 16.47 | 16.65 | 26.06 | 34.35 | 30.35 | 70.55 | 32.06 | 6.92 |
| 120 | 16.70 | 11.68 | 12.99 | 28.25 | 32.53 | 32.40 | 57.74 | 27.47 | 6.06 |
| 140 | 10.96 | 11.40 | 44.50 | 24.48 | 16.43 | 20.88 | 38.80 | 23.92 | 4.96 |
| 160 | 10.03 | 11.31 | 29.76 | 10.35 | 9.90 | 13.96 | 15.97 | 14.47 | 2.69 |
| 180 | 8.98 | 8.06 | 11.20 | 7.76 | 20.97 | 14.36 | 12.57 | 11.99 | 1.76 |

TABLE 5
Individual serum insulin concentration (mU/l) following the third oral
challenge with CHO.

| | Subject | | | | | | | | |
|---|---|---|---|---|---|---|---|---|---|
| Timepoint (min) | S1 | S2 | S6 | S7 | S13 | S15 | S16 | Mean | SEM |
| 0 | 8.55 | 8.48 | 16.05 | 12.83 | 9.13 | 9.75 | 13.66 | 11.21 | 1.12 |
| 15 | 22.67 | 67.90 | 76.25 | 47.01 | 23.55 | 53.15 | 81.51 | 53.15 | 8.99 |
| 30 | 27.09 | 49.77 | 82.14 | 40.74 | 48.90 | 161.09 | 104.18 | 73.42 | 17.65 |
| 45 | 29.02 | 41.97 | 49.91 | 38.67 | 55.73 | 45.59 | 121.81 | 54.67 | 11.64 |
| 60 | 20.60 | 35.93 | 46.79 | 38.84 | 41.73 | 17.07 | 70.99 | 38.85 | 6.76 |
| 80 | 22.03 | 27.91 | 42.49 | 15.91 | 32.96 | 14.50 | 19.97 | 25.11 | 3.79 |
| 100 | 24.71 | 23.88 | 28.90 | 29.35 | 15.89 | 12.17 | 12.57 | 21.07 | 2.80 |
| 120 | 17.26 | 28.02 | 16.93 | 23.81 | 26.05 | 15.58 | 12.10 | 19.96 | 2.26 |
| 140 | 19.88 | 24.36 | 19.65 | 11.16 | 17.81 | 12.64 | 13.61 | 17.02 | 1.79 |
| 160 | 12.52 | 11.79 | 11.54 | 12.19 | 12.93 | 12.06 | 12.91 | 12.28 | 0.20 |
| 180 | 8.68 | 9.85 | 8.90 | 8.21 | 10.92 | 10.25 | 12.09 | 9.84 | 0.52 |

TABLE 6
Individual serum insulin concentration (mU/l) after the first oral
challenge with PAC.

| | Subject | | | | | | | | |
|---|---|---|---|---|---|---|---|---|---|
| Timepoint (min) | S1 | S2 | S6 | S7 | S13 | S15 | S16 | Mean | SEM |
| 0 | 12.07 | 7.31 | 7.90 | 30.34 | 10.00 | 17.68 | 9.33 | 13.52 | 3.10 |
| 15 | 31.31 | 52.09 | 85.42 | 71.26 | 244.68 | 37.16 | 118.24 | 91.45 | 27.93 |
| 30 | 61.12 | 31.53 | 249.46 | 31.37 | 77.93 | 30.86 | 149.09 | 90.19 | 30.96 |
| 45 | 81.41 | 23.65 | 348.91 | 23.17 | 19.15 | 26.67 | 103.82 | 89.54 | 45.04 |
| 60 | 99.74 | 30.97 | 239.66 | 35.81 | 10.43 | 24.72 | 48.15 | 69.93 | 30.25 |
| 80 | 37.20 | 16.95 | 25.04 | 40.41 | 10.99 | 18.60 | 52.32 | 28.79 | 5.64 |
| 100 | 54.74 | 17.34 | 30.13 | 35.72 | 9.96 | 22.62 | 63.76 | 33.47 | 7.43 |
| 120 | 37.17 | 9.74 | 11.36 | 27.50 | 10.84 | 20.71 | 46.63 | 23.42 | 5.44 |
| 140 | 13.97 | 8.79 | 9.99 | 15.94 | 9.81 | 11.72 | 24.16 | 13.48 | 2.02 |
| 160 | 12.12 | 7.39 | 9.47 | 12.60 | 8.84 | 11.30 | 14.54 | 10.89 | 0.93 |
| 180 | 10.00 | 8.22 | 9.25 | 16.95 | 7.74 | 10.30 | 12.22 | 10.67 | 1.19 |

TABLE 7
Individual serum insulin concentration (mU/l) following the third oral
challenge with PAC.

| | Subject | | | | | | | | |
|---|---|---|---|---|---|---|---|---|---|
| Timepoint (min) | S1 | S2 | S6 | S7 | S13 | S15 | S16 | Mean | SEM |
| 0 | 9.90 | 8.34 | 10.60 | 9.68 | 14.75 | 11.81 | 10.80 | 10.84 | 0.77 |
| 15 | 60.82 | 55.50 | 38.59 | 59.98 | 68.13 | 39.25 | 138.10 | 65.77 | 12.76 |
| 30 | 69.06 | 64.98 | 117.88 | 62.21 | 123.53 | 48.56 | 177.51 | 94.82 | 17.56 |
| 45 | 147.14 | 48.80 | 145.18 | 30.83 | 71.60 | 24.44 | 63.69 | 75.96 | 19.18 |
| 60 | 169.42 | 26.13 | 93.38 | 30.21 | 36.91 | 20.58 | 19.65 | 56.61 | 21.13 |
| 80 | 107.83 | 26.69 | 55.08 | 11.86 | 29.62 | 22.49 | 16.54 | 38.59 | 12.68 |
| 100 | 38.48 | 13.39 | 17.65 | 22.94 | 21.51 | 33.58 | 24.17 | 24.53 | 3.31 |
| 120 | 27.83 | 26.65 | 11.74 | 13.30 | 14.30 | 45.78 | 22.82 | 23.21 | 4.50 |
| 140 | 16.83 | 13.28 | 15.76 | 8.41 | 25.41 | 27.15 | 18.84 | 17.95 | 2.49 |
| 160 | 12.18 | 9.61 | 21.46 | 8.14 | 16.39 | 13.66 | 14.20 | 13.66 | 1.67 |
| 180 | 12.45 | 8.60 | 11.06 | 8.15 | 14.18 | 11.12 | 12.27 | 11.12 | 0.81 |

### Serum Insulin Area Under the Concentration Time Curve

TABLE 8
Individual insulin area under the time curve responses (mU/l/min)
following the first oral challenge at 0-180 and 0-80 minutes.

| | 0-180 min | | | 0-80 min | | |
|---|---|---|---|---|---|---|
| | C | CHO | PAC | C | CHO | PAC |
| S1 | −11 | 934 | 3850 | −146 | 1635 | 5954 |
| S2 | −299 | 1810 | 1791 | −714 | 1727 | 2177 |
| S6 | −186 | 1863 | 14129 | −566 | 3552 | 14901 |
| S7 | −32 | 1804 | 718 | −235 | 3363 | 93 |
| S13 | −48 | 1521 | 4693 | −127 | 2932 | 4669 |
| S15 | −50 | 2255 | 757 | −112 | 3566 | 606 |
| S16 | 21 | 4899 | 6256 | −166 | 8290 | 8950 |
| Mean | −86 | 2155 | 4599 | −295 | 3581 | 5336 |
| SE | 114 | 1275 | 4688 | 243 | 2231 | 5248 |

TABLE 9
Individual insulin area under the time curve responses (mU/l/min)
following the third oral challenge at 0-80 and 0-180 minutes.

| | 0-80 | | | 0-180 | | |
|---|---|---|---|---|---|---|
| | C | CHO | PAC | C | CHO | PAC |
| S1 | 10 | 1143 | 7481 | 274 | 2082 | 9600 |
| S2 | −34 | 2688 | 2659 | 1406 | 3978 | 3437 |
| S6 | −18 | 3204 | 5941 | −99 | 3653 | 6875 |
| S7 | 10 | 1805 | 2241 | 274 | 2294 | 2529 |
| S13 | −7 | 2321 | 3821 | −63 | 3301 | 4336 |
| S15 | −23 | 3634 | 1413 | −109 | 3956 | 2972 |
| S16 | 132 | 5064 | 5416 | 195 | 5043 | 6224 |
| Mean | 10 | 2837 | 4139 | 268 | 3472 | 5139 |
| SE | 56 | 1288 | 2213 | 530 | 1028 | 2554 |

### Plasma Creatine Concentration

TABLE 10
Individual plasma creatine concentration (μmol/l) following the first
oral challenge with solution C.

| | Subject | | | | | | | | |
|---|---|---|---|---|---|---|---|---|---|
| Timepoint (min) | S1 | S2 | S6 | S7 | S13 | S15 | S16 | Mean | SEM |
| BSL | 97 | 56 | 15 | 89 | 40 | 59 | 69 | 61 | 11 |
| 15 | 199 | 521 | 221 | 661 | 284 | 564 | 516 | 424 | 70 |
| 30 | 498 | 1090 | 889 | 1139 | 654 | 877 | 742 | 841 | 87 |
| 45 | 721 | 1267 | 316 | 1285 | 554 | 899 | 504 | 792 | 142 |
| 60 | 879 | 1223 | 309 | 1249 | 707 | 906 | 403 | 811 | 138 |
| 80 | 723 | 1099 | 348 | 1157 | 539 | 829 | 327 | 717 | 126 |
| 100 | 863 | 963 | 295 | 961 | 468 | 736 | 267 | 651 | 115 |
| 120 | 707 | 845 | 191 | 788 | 480 | 625 | 208 | 549 | 101 |
| 140 | 683 | 721 | 172 | 603 | 387 | 554 | 172 | 470 | 87 |
| 160 | 465 | 622 | 149 | 560 | 375 | 479 | 145 | 399 | 71 |
| 180 | 591 | 545 | 78 | 507 | 335 | 421 | 126 | 372 | 77 |

TABLE 11
Individual plasma creatine concentration (μmol/l) following the third
oral challenge with solution C.

| | Subject | | | | | | | | |
|---|---|---|---|---|---|---|---|---|---|
| Timepoint (min) | S1 | S2 | S6 | S7 | S13 | S15 | S16 | Mean | SEM |
| BSL | 65 | 136 | 38 | 150 | 79 | 129 | 54 | 93 | 17 |
| 15 | 583 | 586 | 285 | 708 | 202 | 599 | 464 | 490 | 70 |
| 30 | 1243 | 930 | 482 | 1412 | 502 | 778 | 680 | 861 | 135 |
| 45 | 1538 | 957 | 375 | 1389 | 508 | 802 | 620 | 884 | 167 |
| 60 | 1506 | 898 | 265 | 1287 | 636 | 727 | 561 | 840 | 163 |
| 80 | 1210 | 993 | 290 | 1167 | 554 | 640 | 446 | 757 | 138 |
| 100 | 826 | 662 | 240 | 944 | 522 | 1006 | 372 | 653 | 110 |
| 120 | 782 | 584 | 151 | 768 | 533 | 976 | 318 | 588 | 108 |
| 140 | 953 | 509 | 173 | 481 | 482 | 481 | 293 | 482 | 92 |
| 160 | 739 | 454 | 142 | 608 | 402 | 440 | 239 | 432 | 77 |
| 180 | 753 | 392 | 101 | 493 | 418 | 612 | 211 | 426 | 84 |

TABLE 12
Individual plasma creatine concentration (μmol/l) following the first
oral challenge with solution CHO.

| | Subject | | | | | | | | |
|---|---|---|---|---|---|---|---|---|---|
| Timepoint (min) | S1 | S2 | S6 | S7 | S13 | S15 | S16 | Mean | SEM |
| BSL | 40 | 76 | 34 | 36 | 59 | 72 | 37 | 51 | 7 |
| 15 | 63 | 142 | 57 | 75 | 76 | 93 | 66 | 82 | 11 |
| 30 | 165 | 349 | 119 | 165 | 116 | 145 | 173 | 176 | 30 |
| 45 | 275 | 728 | 194 | 258 | 192 | 283 | 275 | 315 | 70 |
| 60 | 381 | 904 | 260 | 458 | 292 | 375 | 365 | 433 | 82 |
| 80 | 522 | 1140 | 414 | 564 | 339 | 497 | 505 | 569 | 99 |
| 100 | 665 | 1311 | 659 | 784 | 386 | 620 | 603 | 718 | 109 |
| 120 | 745 | 1309 | 536 | 723 | 504 | 657 | 646 | 731 | 102 |
| 140 | 691 | 1288 | 547 | 780 | 496 | 762 | 660 | 746 | 99 |
| 160 | 645 | 1193 | 603 | 768 | 484 | 655 | 538 | 698 | 89 |
| 180 | 520 | 686 | 610 | 616 | 545 | 542 | 475 | 571 | 27 |

TABLE 13
Individual plasma creatine concentration (μmol/l) following the third
oral challenge with solution CHO.

| | Subject | | | | | | | | |
|---|---|---|---|---|---|---|---|---|---|
| Timepoint (min) | S1 | S2 | S6 | S7 | S13 | S15 | S16 | Mean | SEM |
| BSL | 65 | 107 | 88 | 78 | 95 | 68 | 69 | 81 | 6 |
| 15 | 95 | 141 | 255 | 104 | 118 | 86 | 98 | 128 | 22 |
| 30 | 125 | 566 | 166 | 274 | 152 | 214 | 216 | 245 | 57 |
| 45 | 289 | 794 | 330 | 509 | 194 | 318 | 316 | 393 | 76 |
| 60 | 356 | 1105 | 448 | 623 | 331 | 308 | 424 | 514 | 106 |
| 80 | 385 | 1271 | 657 | 637 | 469 | 313 | 445 | 597 | 122 |
| 100 | 635 | 1363 | 697 | 823 | 563 | 299 | 392 | 682 | 132 |
| 120 | 758 | 1432 | 756 | 892 | 670 | 284 | 367 | 737 | 143 |
| 140 | 842 | 1528 | 801 | 1023 | 745 | 251 | 352 | 792 | 161 |
| 160 | 868 | 1231 | 739 | 745 | 705 | 233 | 322 | 692 | 127 |
| 180 | 726 | 1342 | 592 | 726 | 599 | 195 | 315 | 642 | 139 |

TABLE 14
Individual plasma creatine concentration (μmol/l) following the first
oral challenge with solution PAC.

| | Subject | | | | | | | | |
|---|---|---|---|---|---|---|---|---|---|
| Timepoint (min) | S1 | S2 | S6 | S7 | S13 | S15 | S16 | Mean | SEM |
| BSL | 85 | 63 | 71 | 54 | 35 | 65 | 37 | 59 | 7 |
| 15 | 96 | 150 | 217 | 88 | 103 | 100 | 131 | 126 | 17 |
| 30 | 233 | 442 | 431 | 217 | 141 | 277 | 284 | 289 | 42 |
| 45 | 780 | 613 | 662 | 340 | 168 | 493 | 505 | 509 | 78 |
| 60 | 785 | 770 | 680 | 444 | 151 | 543 | 404 | 540 | 86 |
| 80 | 653 | 921 | 591 | 589 | 136 | 569 | 442 | 557 | 89 |
| 100 | 947 | 966 | 708 | 708 | 186 | 567 | 558 | 663 | 101 |
| 120 | 405 | 918 | 968 | 767 | 223 | 619 | 555 | 636 | 102 |
| 140 | 602 | 748 | 515 | 674 | 180 | 537 | 462 | 531 | 69 |
| 160 | 533 | 628 | 630 | 605 | 179 | 486 | 417 | 497 | 61 |
| 180 | 490 | 540 | 553 | 558 | 106 | 388 | 346 | 426 | 62 |

TABLE 15
Individual plasma creatine concentration (μmol/l) following the third
oral challenge with solution PAC.

| | Subject | | | | | | | | |
|---|---|---|---|---|---|---|---|---|---|
| Timepoint (min) | S1 | S2 | S6 | S7 | S13 | S15 | S16 | Mean | SEM |
| BSL | 136 | 140 | 136 | 105 | 63 | 65 | 90 | 105 | 13 |
| 15 | 245 | 304 | 125 | 180 | 62 | 86 | 193 | 171 | 33 |
| 30 | 267 | 629 | 257 | 414 | 180 | 207 | 313 | 324 | 58 |
| 45 | 471 | 830 | 459 | 600 | 242 | 324 | 487 | 488 | 72 |
| 60 | 656 | 870 | 848 | 782 | 263 | 392 | 450 | 609 | 91 |
| 80 | 680 | 944 | 1107 | 698 | 279 | 453 | 509 | 667 | 108 |
| 100 | 755 | 861 | 1009 | 831 | 314 | 557 | 549 | 697 | 89 |
| 120 | 802 | 994 | 934 | 957 | 251 | 660 | 529 | 732 | 103 |
| 140 | 747 | 965 | 1021 | 764 | 439 | 701 | 532 | 738 | 80 |
| 160 | 988 | 812 | 1139 | 668 | 293 | 707 | 496 | 729 | 108 |
| 180 | 512 | 649 | 930 | 708 | 275 | 567 | 480 | 589 | 77 |

### Plasma Creatine Area Under the Concentration Time Curve

TABLE 16
Individual plasma creatine area under the time curve responses
(μmol/l/min) following the first oral challenge
at 0-80 and 0-180 minutes.

| | 0-80 min | | | 0-180 min | | |
|---|---|---|---|---|---|---|
| | C | CHO | PAC | C | CHO | PAC |
| S1 | 36856 | 16556 | 30754 | 94686 | 77948 | 83425 |
| S2 | 71527 | 39970 | 36199 | 14533 | 152639 | 109728 |
| S6 | 29206 | 11739 | 32283 | 48115 | 65438 | 93032 |
| S7 | 73234 | 18476 | 19419 | 139163 | 87738 | 80584 |
| S13 | 37231 | 9961 | 7623 | 76151 | 50268 | 21867 |
| S15 | 54988 | 14110 | 23516 | 109504 | 71120 | 70771 |
| S16 | 31757 | 16469 | 22594 | 45215 | 71512 | 66593 |
| Mean | 47828 | 18183 | 24627 | 75338 | 82380 | 75143 |
| SE | 7065 | 3799 | 3624 | 16155 | 12486 | 10402 |

TABLE 17
Individual plasma creatine area under the time curve
responses (μmol/l/min) following the third oral
challenge at 0-180 and 0-80 minutes.

| | 0-80 min | | | 0-180 min | | |
|---|---|---|---|---|---|---|
| | C | CHO | PAC | C | CHO | PAC |
| S1 | 84202 | 36570 | 23142 | 163321 | 99883 | 87286 |
| S2 | 52861 | 64664 | 40942 | 97237 | 171193 | 115450 |
| S6 | 21862 | 19314 | 28665 | 36055 | 82891 | 114359 |
| S7 | 75957 | 24914 | 30932 | 133599 | 29975 | 98890 |
| S13 | 29085 | 10536 | 10099 | 69617 | 65335 | 35275 |
| S15 | 42432 | 12863 | 15944 | 100109 | 32523 | 72164 |
| S16 | 36837 | 16344 | 21356 | 62497 | 45732 | 64384 |
| Mean | 49034 | 26458 | 24440 | 94634 | 75362 | 83972 |
| SE | 8871 | 7165 | 3846 | 16448 | 18718 | 10957 |

### Urinary Creatine Excretion

TABLE 18
Individual urinary creatine content (mg) for 0-24, 24-48 and 0-48
hours following administration of solutions C, CHO, and PAC.

| | 0-24 h | | | 24-48 h | | | 0-48 h | | |
|---|---|---|---|---|---|---|---|---|---|
| | C | CHO | PAC | C | CHO | PAC | C | CHO | PAC |
| S1 | 6388 | 2816 | 4284 | 4163 | 0 | 2190 | 10551 | 2816 | 6474 |
| S2 | 7117 | 5731 | 4447 | 0 | 313 | 1379 | 7117 | 6044 | 5826 |
| S6 | 8035 | 6090 | 6485 | 1001 | 1325 | 133 | 9036 | 7415 | 6618 |
| S7 | 6762 | 5489 | 3289 | 0 | 1224 | 0 | 6762 | 6713 | 3289 |
| S13 | 7437 | 5812 | 2800 | 3648 | 232 | 0 | 11084 | 6043 | 2800 |
| S15 | 5291 | 3199 | 3863 | 840 | 0 | 563 | 6132 | 3163 | 4427 |
| S16 | 5626 | 6073 | 6630 | 0 | 804 | 1750 | 5626 | 6877 | 8380 |
| Mean | 6665 | 5030 | 4543 | 1379 | 557 | 859 | 8044 | 5582 | 5402 |
| SE | 369 | 530 | 562 | 673 | 212 | 342 | 824 | 694 | 754 |

We claim:

1. A low carbohydrate creatine supplement comprising: a creatine, a carbohydrate source, a protein source and one or more naturally occurring free amino acids, wherein a serving of the low carbohydrate creatine supplement is effective in amplifying creatine accumulation in skeletal muscle.

2. The supplement of claim 1, wherein a serving of the low carbohydrate creatine supplement comprises less than about 30 calories derived from the carbohydrate source, the protein source and the naturally occurring free amino acids per gram of creatine.

3. The supplement of claim 1, wherein the naturally occurring free amino acid is selected from the group consisting of L-Leucine and L-Phenylalanine.

4. A method for activating muscle gene expression including the step of administering a low carbohydrate creatine supplement comprising: a creatine, a carbohydrate source, a protein source and one or more naturally occurring free amino acids, wherein a serving of the low carbohydrate creatine supplement is effective in amplifying creatine accumulation in skeletal muscle.

5. The method of claim 4, wherein the serving stimulates gene expression for muscle growth.

6. The method of claim 4, wherein the serving turns on muscle promoting pathways.

7. The method of claim 4, wherein the serving stimulates muscle growth.

8. The method of claim 4, wherein the serving accelerates muscle protein synthesis.

9. The method of claim 4, wherein the serving activates mTOR expression to turn on protein synthesis.

10. The method of claim 4, wherein a serving of the low carbohydrate creatine supplement comprises less than about 30 calories derived from the carbohydrate source, the protein source and the naturally occurring free amino acids per gram of creatine.

11. The method of claim 4, wherein the naturally occurring free amino acid is selected from the group consisting of L-Leucine and L-Phenylalanine.

12. A method for switching off catabolism in skeletal muscle of a user, including the step of administering a low carbohydrate creatine supplement comprising: a creatine, a carbohydrate source, a protein source and one or more naturally occurring free amino acids, wherein a serving of the low carbohydrate creatine supplement is effective in amplifying creatine accumulation in skeletal muscle.

13. The method of claim 12, wherein the serving stimulates gene expression for muscle growth.

14. The method of claim 12, wherein the serving turns on muscle promoting pathways.

15. The method of claim 12, wherein the serving stimulates muscle growth.

16. The method of claim 12, wherein the serving accelerates muscle protein synthesis.

17. The method of claim 12, wherein the serving activates mTOR expression to turn on protein synthesis.

18. The method of claim 12, wherein a serving of the low carbohydrate creatine supplement comprises less than about 30 calories derived from the carbohydrate source, the protein source and the naturally occurring free amino acids per gram of creatine.

19. The method of claim 12, wherein the naturally occurring free amino acid is selected from the group consisting of L-Leucine and L-Phenylalanine.

20. A method for reaching maximum protein synthesis rates in skeletal muscle of a user, including the step of administering a low carbohydrate creatine supplement comprising: a creatine, a carbohydrate source, a protein source and one or more naturally occurring free amino acids, wherein a serving of the low carbohydrate creatine supplement is effective in amplifying creatine accumulation in skeletal muscle.

21. The method of claim 20, wherein the serving stimulates gene expression for muscle growth.

22. The method of claim 20, wherein the serving turns on muscle promoting pathways.

23. The method of claim 4, wherein the serving stimulates muscle growth.

24. The method of claim 20, wherein the serving accelerates muscle protein synthesis.

25. The method of claim 20, wherein the serving activeates mTOR expression to turn on protein synthesis.

26. The method of claim 20, wherein a serving of the low carbohydrate creatine supplement comprises less than about 30 calories derived from the carbohydrate source, the protein source and the naturally occurring free amino acids per gram of creatine.

27. The method of claim 20, wherein the naturally occurring free amino acid is selected from the group consisting of L-Leucine and L-Phenylalanine.

28. A method for genetically enhancing the building of muscle in a user, including the step of administering a low carbohydrate creatine supplement comprising: a creatine, a carbohydrate source, a protein source and one or more naturally occurring free amino acids, wherein a serving of the low carbohydrate creatine supplement is effective in amplifying creatine accumulation in skeletal muscle.

29. The method of claim 28, wherein the serving stimulates gene expression for muscle growth.

30. The method of claim 28, wherein the serving turns on muscle promoting pathways.

31. The method of claim 28, wherein the serving stimulates muscle growth.

32. The method of claim 28, wherein the serving accelerates muscle protein synthesis.

33. The method of claim 28, wherein the serving activeates mTOR expression to turn on protein synthesis.

34. The method of claim 28, wherein a serving of the low carbohydrate creatine supplement comprises less than about 30 calories derived from the carbohydrate source, the protein source and the naturally occurring free amino acids per gram of creatine.

35. The method of claim 28, wherein the naturally occurring free amino acid is selected from the group consisting of L-Leucine and L-Phenylalanine.

36. A method for manufacturing a low carbohydrate creatine supplement comprising the step of mixing a creatine, a carbohydrate source, a protein source and a naturally occurring free amino acid;
blending and mixing for 30 minutes; and
checking for uniformity/homogeneity and then aliquoting into a serving.
